SEQUENCE LISTING (40 sequences)

SEQ ID NO: 1 - DNA, 44 bp, Lactuca sativa
  source 1..44: mol_type = other DNA
gaaggtgaccaagttcatgctttaaaaacacacgctgagtgcac

SEQ ID NO: 2 - DNA, 47 bp, Lactuca sativa
  source 1..47: mol_type = other DNA
gaaggtcggagtcaacggattcctttaaaaacacacgctgagtgcat

SEQ ID NO: 3 - DNA, 30 bp, Lactuca sativa
  source 1..30: mol_type = other DNA
ctaaggatttttcaacaaattttgtgggat

SEQ ID NO: 4 - DNA, 29 bp, Lactuca sativa
  source 1..29: mol_type = other DNA
gtggggaggtttaggaattatgtttgatt

SEQ ID NO: 5 - DNA, 200 bp, Lactuca sativa
  source 1..200: mol_type = genomic DNA
gaatctaaggaaagtggagttggtggggaggtttaggaattatgtttgatttttttaggaattctaaggatttttcaacaaattttgtgggattttagartgcactcagcgtgtgtttttaaaggtacttttttttgttttttctttaaaggtggtattatgtcatccttcatttagactcaattgttccaaagcttctt

SEQ ID NO: 6 - DNA, 54 bp, Lactuca sativa
  source 1..54: mol_type = other DNA
gaaggtgaccaagttcatgctataaaaatacaaactacgctacacttattttgt

SEQ ID NO: 7 - DNA, 51 bp, Lactuca serriola
  source 1..51: mol_type = other DNA
gaaggtcggagtcaacggattaaaatacaaactacgctacacttattttgg

SEQ ID NO: 8 - DNA, 30 bp, Lactuca sativa
  source 1..30: mol_type = other DNA
gttttagtcttaaaccctaagactggagtt

SEQ ID NO: 9 - DNA, 29 bp, Lactuca sativa
  source 1..29: mol_type = other DNA
gagtgccccccaaaaaatcctatatttta

SEQ ID NO: 10 - DNA, 200 bp, Lactuca sativa
  source 1..200: mol_type = genomic DNA
tccttgtcagtaattataaagagagtgccccccaaaaaatcctatattttatgttatacgaatagttttagtcttaaaccctaagactggagttagtgtmcaaaataagtgtagcgtagtttgtatttttatgaaaacatgtagagtgtgtattcttgtaaattcaaaataatgttgttattccttatgtgaagttacaa

SEQ ID NO: 11 - DNA, 53 bp, Lactuca sativa
  source 1..53: mol_type = other DNA
gaaggtgaccaagttcatgcttctattggtttacattctatatagtgttgttt

SEQ ID NO: 12 - DNA, 52 bp, Lactuca sativa
  source 1..52: mol_type = other DNA
gaaggtcggagtcaacggattctattggtttacattctatatagtgttgttc

SEQ ID NO: 13 - DNA, 28 bp, Lactuca sativa
  source 1..28: mol_type = other DNA
tcaatttcgagccaatcaagtgggaaaa

SEQ ID NO: 14 - DNA, 29 bp, Lactuca sativa
  source 1..29: mol_type = other DNA
aaccatctactcaatttcgagccaatcaa

SEQ ID NO: 15 - DNA, 200 bp, Lactuca sativa
  source 1..200: mol_type = genomic DNA
agaatgaaaacgaaaggattaccaaaatattccacgacatttcaaatttctcttcttcaaaaccatctactcaatttcgagccaatcaagtgggaaaacraacaacactatatagaatgtaaaccaatagaaagagcatatagcatggagataactacagtttgtaacacaagaactagcaagaacagagattaaatcaa

SEQ ID NO: 16 - DNA, 47 bp, Lactuca sativa
  source 1..47: mol_type = other DNA
gaaggtgaccaagttcatgctacgtcaaaatggtccttcaagaacat

SEQ ID NO: 17 - DNA, 47 bp, Lactuca sativa
  source 1..47: mol_type = other DNA
gaaggtcggagtcaacggattacgtcaaaatggtccttcaagaacaa

SEQ ID NO: 18 - DNA, 29 bp, Lactuca sativa
  source 1..29: mol_type = other DNA
ggttattctctctacgaatgaagaaggta

SEQ ID NO: 19 - DNA, 29 bp, Lactuca sativa
  source 1..29: mol_type = other DNA
ttgaagcaacggttattctctctacgaat

SEQ ID NO: 20 - DNA, 200 bp, Lactuca sativa
  source 1..200: mol_type = genomic DNA
atatgcgagtgatgttttacctacacttggccatccagagtgtttccataaggtttgcggcaacttggagtaacacgtcaaaatggtccttcaagaacawtaccttcttcattcgtagagagaataaccgttgcttcaatagtaattttttcgttatgaagtttgtcttgtacagactctcaagcgtgaaccatagagac

SEQ ID NO: 21 - DNA, 40 bp, Lactuca sativa
  source 1..40: mol_type = other DNA
gaaggtgaccaagttcatgctttccagaaccaccacgccc

SEQ ID NO: 22 - DNA, 42 bp, Lactuca sativa
  source 1..42: mol_type = other DNA
gaaggtcggagtcaacggattctttccagaaccaccacgcct

SEQ ID NO: 23 - DNA, 29 bp, Lactuca sativa
  source 1..29: mol_type = other DNA
ggagcaacaaacaactttgctgcaaaata

SEQ ID NO: 24 - DNA, 29 bp, Lactuca sativa
  source 1..29: mol_type = other DNA
gaagagataagaggagcaacaaacaactt

SEQ ID NO: 25 - DNA, 200 bp, Lactuca sativa
  source 1..200: mol_type = genomic DNA
aaacaaatcatgtcgttcctggaaaagtttggtcacttaagaattccactggaagagataagaggagcaacaaacaactttgctgcaaaatatttaattrggcgtggtggttctggaaagatctacaaaggagtgctcatccggccaggaggctctgtcagggtcgcattcaagttgcttgatagagcattcgtccaagg

SEQ ID NO: 26 - DNA, 51 bp, Lactuca sativa
  source 1..51: mol_type = other DNA
gaaggtgaccaagttcatgctggttccatattgaaaaattcaactcatcta

SEQ ID NO: 27 - DNA, 51 bp, Lactuca sativa
  source 1..51: mol_type = other DNA
gaaggtcggagtcaacggattggttccatattgaaaaattcaactcatctt

SEQ ID NO: 28 - DNA, 29 bp, Lactuca sativa
  source 1..29: mol_type = other DNA
gttgcacaatatgtggactatttgcctta

SEQ ID NO: 29 - DNA, 30 bp, Lactuca sativa
  source 1..30: mol_type = other DNA
gacttacaaagttgcacaatatgtggacta

SEQ ID NO: 30 - DNA, 200 bp, Lactuca sativa
  source 1..200: mol_type = genomic DNA
aatgactattcatgtacatagtaggggtttcactttcacaagtgcttgtagtattcacaacaatcaatttggttccatattgaaaaattcaactcatctwttttaaggcaaatagtccacatattgtgcaactttgtaagtctgtttacaacccaagacatgaatgcaatttgttcattaatgtagtgttataggctatt

SEQ ID NO: 31 - DNA, 47 bp, Lactuca sativa
  source 1..47: mol_type = other DNA
gaaggtgaccaagttcatgctttagacgaggtaacacaagtatctct

SEQ ID NO: 32 - DNA, 45 bp, Lactuca sativa
  source 1..45: mol_type = other DNA
gaaggtcggagtcaacggattagacgaggtaacacaagtatctcc

SEQ ID NO: 33 - DNA, 29 bp, Lactuca sativa
  source 1..29: mol_type = other DNA
gagaaagaatctgatggcaagacgaataa

SEQ ID NO: 34 - DNA, 29 bp, Lactuca sativa
  source 1..29: mol_type = other DNA
gaagacaaagagaaagaatctgatggcaa

SEQ ID NO: 35 - DNA, 200 bp, Lactuca sativa
  source 1..200: mol_type = genomic DNA
taatgggaatgaaaaatcctccttccccaagctaaaccccttaagacatggaagccattctaatgtcaaggactttagacgaggtaacacaagtatctcyttattcgtcttgccatcagattctttctctttgtcttcttctacacaaacatctgcatccttaacaatcacctcctccatttgtctgcagtaatctatat

SEQ ID NO: 36 - DNA, 49 bp, Lactuca sativa
  source 1..49: mol_type = other DNA
gaaggtgaccaagttcatgctgattctttctctttgtcttcttctacac

SEQ ID NO: 37 - DNA, 45 bp, Lactuca sativa
  source 1..45: mol_type = other DNA
gaaggtcggagtcaacggattctttctctttgtcttcttctacag

SEQ ID NO: 38 - DNA, 28 bp, Lactuca sativa
  source 1..28: mol_type = other DNA
gaggtgattgttaaggatgcagatgttt

SEQ ID NO: 39 - DNA, 28 bp, Lactuca sativa
  source 1..28: mol_type = other DNA
acaaatggaggaggtgattgttaaggat

SEQ ID NO: 40 - DNA, 200 bp, Lactuca sativa
  source 1..200: mol_type = genomic DNA
catggaagccattctaatgtcaaggactttagacgaggtaacacaagtatctctttattcgtcttgccatcagattctttctctttgtcttcttctacasaaacatctgcatccttaacaatcacctcctccatttgtctgcagtaatctatatgtagctcttggagttgcgatagactaccaaccatggaactagtaaa

(19) United States
(12) Patent Application Publication
(10) Pub. No.: US 2025/0081912 A1
(43) Pub. Date: Mar. 13, 2025

(54) LETTUCE LINE HAVING RESISTANCE TO FUSARIUM WILT

(71) Applicant: SOUTHWEST GENETICS, LLC, YUMA, AZ (US)

(72) Inventors: DAVIE LEE BROOKS, II, YUMA, AZ (US); JON CODY NICKERSON, YUMA, AZ (US)

(21) Appl. No.: 18/883,531

(22) Filed: Sep. 12, 2024

Related U.S. Application Data
(60) Provisional application No. 63/537,894, filed on Sep. 12, 2023.

(51) Int. Cl. A01H 6/14; A01H 1/00; A01H 5/12

(52) U.S. Cl. CPC A01H 6/1472; A01H 1/1255; A01H 5/12

(57) ABSTRACT

The present invention is directed to lettuce lines having resistance to Fusarium wilt. The invention relates to the seeds, plants, and plant parts of these lettuce lines. The invention further relates to methods for producing a lettuce plant which produces seeds having a resistance to Fusarium wilt containing in its genetic material one or more transgenes and to the transgenic plants produced by those methods. This invention also relates to lettuce cultivars or breeding cultivars and plant parts derived from lettuce plants which produce seeds having resistance to Fusarium wilt and to methods for producing other lettuce cultivars, lines or plant parts derived from lettuce plants which produce seeds having resistance to Fusarium wilt.

### CROSS-REFERENCE TO RELATED APPLICATION

The present application is related to and claims priority to U.S. Patent Application Ser. No. 63/537,894, filed on Sep. 12, 2023, which is incorporated herein in its entirety.

### BACKGROUND OF THE INVENTION

The present invention relates to new resistant lettuce products and Fusarium wilt resistant plant lines, including lettuce plants, seeds, varieties, and hybrids. The invention further relates to lettuce seed having a resistance to Fusarium wilt and to novel lettuce plants and lines which produce seeds having a resistance to Fusarium wilt. The invention further relates to plants, seeds, plant parts and tissue cultures of the novel lettuce lines described herein further comprising introduced beneficial or desirable traits. All of the publications cited in this application are herein incorporated by reference in their entirety for all that they disclose.

Lettuce is an important vegetable crop grown around the world. Fusarium wilt of lettuce can result in substantial reductions in crop yield potential. Symptoms of Fusarium wilt include yellowing of leaves, wilting, vascular discoloration, root rot, and plant death. Current management practices focus primarily on keeping inoculum soil levels low and using varieties with some lettuce types. Current varieties of lettuce do not have an adequate level of Fusarium wilt resistance to prevent significant reduction in yield.

Fusarium wilt of lettuce is caused by the fungal pathogen Fusarium oxysporum f.sp. lactucae. Although it can colonize other hosts, this pathogen is only known to cause disease in lettuce. It was first documented in the U.S. in 1990 in California and appeared shortly after in Arizona. Fusarium wilt of lettuce was first reported in Florida in 2017. Four races of this pathogen have been identified, but only Race 1 has been found in the U.S Citations 1 and 2 set forth below.

The symptoms of Fusarium wilt can vary depending on the lettuce variety's susceptibility, the level of inoculum in the soil, and the air and soil temperatures. The fungus infects through secondary roots and colonizes the taproot's xylem (water conducting tissue). Xylem infection slows the flow of water and nutrients to the upper plant, and above-ground symptoms result from the reduced availability of water and nutrients.

Above-ground symptoms include wilting of leaves, stunting, and chlorosis (yellowing), eventually leading to leaf necrosis and plant death. Citations 2 and 3 set forth below. Stunting can be severe, and affected plants may fail to produce heads. On older plants, the yellowing of leaves can be accompanied by tip burn, and a brown to black discoloration of the leafs vascular tissue can develop. Citation 4 set forth below. Few or no symptoms may develop on the above ground plant parts even when the root system is heavily infected. 3 Seedlings can also wilt and die. Infected roots typically show a reddish-brown discoloration of the cortex of the crown and upper tissues of the taproot. The core of the taproot can be extensively rotted. Citations 2 and 4 set forth below.

Symptoms of Fusarium wilt are similar to several other diseases, including Verticillium wilt, lettuce drop caused by Sclerotinia sclerotiorum and S. minor, gray mold, bacterial soft rot, and corky root rot. All of these diseases can result in stunting and plant collapse. The reddish-brown discoloration of the taproot tissue is one of the most distinguishing features of Fusarium wilt on lettuce. Citations 2, 3, and 4 set forth below.

The Fusarium wilt pathogen is a soilborne fungus distributed in infested soil and crop debris. Inoculum can be moved by wind, flowing water, and on tools and equipment. Citations 1 and 2 set forth below. Spores of the fungus have a half-life in the soil of about six months. However, once introduced into a field, the pathogen will most likely remain in the field indefinitely, surviving on the roots of various crop and weed plant species. Fusarium oxysporum f.sp. lactucae has been shown to colonize the roots of broccoli, cauliflower, and spinach, which are common rotational crops for lettuce. The level of colonization of these three crops varies, with broccoli being a fairly poor host. The pathogen can colonize cauliflower roots somewhat better, but the fungus was isolated from 50% of spinach plants exposed to inoculum. Citations 1 and 5 set forth below. The pathogen can also extensively colonize the root systems of some lettuce varieties characterized as resistant to Fusarium wilt. The severity of above-ground symptoms is not always well correlated with the level of root colonization. This means that the inoculum levels of the Fusarium wilt pathogen can increase in a field planted with a resistant variety of lettuce. Citation 5 set forth below. Local spread is mainly through the movement of infested soil or crop debris. Long-distance distribution is thought to be primarily through infested seeds. Citations 1 and 3 set forth below.

Disease development and symptom expression are affected by inoculum density and temperature. Therefore, higher levels of the pathogen in the soil are correlated with higher disease incidence and severity. Symptoms are also more severe during periods with high temperatures (91/72° F. day/night) as compared to low-temperature periods (74/64° F. day/night). Citations 1 and 3 set forth below. In California, Fusarium wilt on lettuce crops planted in June has been more severe than on crops planted in July and August because of the temperatures that occur during the primary stages of production.

Current management is to avoid planting susceptible lettuce varieties in fields with a history of Fusarium wilt for at least three years after the last lettuce crop. Avoid introducing the pathogen into non-infested fields by using only disease-free seeds and proper sanitation practices. Do not use seeds that are contaminated with soil or crop debris. Chemical seed treatments may provide some protection against seedborne inoculum. Clean equipment and tools between fields to prevent the movement of soil between fields. It can take several years for the pathogen to build up to levels where symptoms become apparent, so cleaning equipment after use, even in fields with no history of the disease, is recommended.

For fields with a history of Fusarium wilt on lettuce, several strategies can help lower inoculum levels. One strategy is soil solarization, a process in which soils are covered with clear plastic to capture solar radiation and increase the temperature of the soil. Solarization is typically done during the warmest time of the year. Citations 1 and 3 set forth below. A study performed in Arizona in 2010 found that soil solarization reduced inoculum levels of the lettuce Fusarium wilt pathogen by 91%. In that study, soil temperatures at a depth of two inches were raised to as high as 120° F. (49° C.). Soil fumigation is a strategy used to help manage Fusarium wilt in other crops, such as lettuce, and it might help manage the disease on lettuce. However, soil fumigation is expensive and not economically feasible for most lettuce production operations. Fallowing fields can also help lower the inoculum level of the wilt pathogen, but the practice will probably not result in the complete elimination of the pathogen. One study showed a rapid decline in the level of Fusarium oxysporum f.sp. lactucae in the first year of fallowing but a slower decline thereafter. The pathogen was still detectable in the soil two and a half years after the last lettuce crop. In fields where Fusarium wilt has been identified, rotate to non-host crops for at least three years. Rotations should not include brassicas or spinach, and resistant lettuce varieties should not be considered as a non-host crop. Citations 1, 2 and 4 set forth below.

Time plantings to avoid having the primary production period during the warmest times of the year. It is especially important to grow the most susceptible lettuce varieties during the coolest parts of the growing season. Citations 1, 2 and 3 set forth below. Some lettuce varieties are characterized as having resistance to Fusarium wilt. Resistance levels are present in some leaf and romaine lettuce varieties. Citations 1 and 3 set forth below. The evaluation of resistance to Fusarium wilt is sometimes based only on the expression of symptoms on above-ground parts of the lettuce plant, not on symptoms on the roots or levels of pathogen colonization of the roots. Citation 3 set forth below. Selecting resistant varieties that also show lower colonization of the root system helps to minimize the buildup of pathogen inoculum when lettuce crops are grown. Currently, only Race 1 of the Fusarium wilt pathogen has been found in the U.S., but Races 2, 3, and 4 are present in other areas of the world. Taking steps to minimize the long-distance dispersal of the Fusarium wilt pathogen will help prevent the introduction of new races and protect the effectiveness of the resistance genes available in some lettuce varieties. Citation 3 set forth below.
Citation 1: Gordon, T. and Koike, S. 2017. Fusarium wilt. In: Compendium of Lettuce Diseases and Pests, 2nd Ed. Subbarao, K., Davis, M., Gilbertson, R., and Raid, R. eds. The American Phytopathological Society, St Paul.
Citation 2: Sandoya, G. Murray, J., Raid, R., and Miller, C. 2020. Fusarium wilt: a new threat to Florida lettuce production. UF-IFAS Extension. University of Florida, publication HS1385. https colon slash slash edis period ifas.ufl.edu/publication/HS1385.
Citation 3: Gordon, T. and Koike, S. 2015. Management of Fusarium wilt of lettuce. Crop Protection 73:45-49.
Citation 4: Koike, S. and Turini, T. 2017. Fusarium wilt. UC-IPM. Lettuce Pest Management Guidelines. https://www2.ipm.ucanr.edu/agriculture/lettuce/fusarium-wilt/.
Citation 5: Scott, J. C., McRoberts, D. N., and Gordon, T. R. 2014. Colonization of lettuce cultivars and rotation crops by Fusarium oxysporum f. sp. lactucae, the cause of Fusarium wilt of lettuce. Plant Pathology 63:548-553.
Citation 6 Matheron, M. and Porchas, M. 2010. Evaluation of soil solarization and flooding as management tools for Fusarium wilt of lettuce. Plant Disease 94:1323-1328.

The goal of vegetable breeding is to combine various desirable traits in a single variety. Such desirable traits may include any trait deemed beneficial or desirable by a grower or consumer, including greater yield, resistance to insects or disease, tolerance to environmental stress, and nutritional value.

Breeding techniques take advantage of a plant's method of pollination. There are two general methods of pollination: a plant self-pollinates if pollen from one flower is transferred to the same or another flower of the same plant or plant variety. A plant cross-pollinates if pollen comes to it from a flower of a different plant variety.

Plants that have been self-pollinated and selected for type over many generations become homozygous at almost all genetic loci and produce a uniform population of true breeding progeny, a homozygous plant. A cross between two such homozygous plants of different genotypes produces a uniform population of hybrid plants that are heterozygous for many genetic loci. Conversely, a cross of two plants each heterozygous at a number of loci produces a population of hybrid plants that differ genetically and are not uniform. The resulting non-uniformity makes performance unpredictable.

The development of uniform varieties requires the development of homozygous inbred plants, the crossing of these inbred plants, and the evaluation of the crosses. Pedigree breeding and recurrent selection are examples of breeding methods that have been used to develop inbred plants from breeding populations. Those breeding methods combine the genetic backgrounds from two or more plants or various other broad-based sources into breeding pools from which new lines and hybrids derived therefrom are developed by selfing and selection of desired phenotypes. The new lines and hybrids are evaluated to determine which of those have commercial potential.

The foregoing examples of the related art and limitations related therewith are intended to be illustrative and not exclusive. Other limitations of the related art will become apparent to those of skill in the art upon a reading of the specification.

### SUMMARY OF THE INVENTION

The following embodiments and aspects thereof are described in conjunction with systems, tools and methods which are meant to be exemplary and illustrative, not limiting in scope. In various embodiments, one or more of the above-described problems, such as susceptibility to Fusarium wilt, have been reduced or eliminated, while other embodiments are directed to other improvements.

In one aspect, the present invention provides a lettuce plant having resistance to Fusarium wilt. In one embodiment, the lettuce plant is of line ADRIAN, MICKEY, BALBOA, or PAULIE. Also provided are lettuce plants having all the physiological and morphological characteristics of such a plant. Parts of these lettuce plants are also provided, for example, pollen, an ovule, an embryo, a seed, a scion, a rootstock, a fruit, and a cell of the plant.

The invention further provides methods for developing lettuce plants derived from lettuce plants that produce seed having resistance to Fusarium wilt, including but not limited to lettuce cultivar ADRIAN, MICKEY, BALBOA, or PAULIE, in a lettuce plant breeding program using plant breeding techniques including recurrent selection, backcrossing, pedigree breeding, restriction fragment length polymorphism enhanced selection, genetic marker enhanced selection, and transformation. Seeds, lettuce plants, and parts thereof, produced by such breeding methods are also part of the invention.

In another aspect of the invention, a plant of lettuce line ADRIAN, MICKEY, BALBOA, or PAULIE, comprising an added heritable trait is provided. The heritable trait may comprise a genetic locus that is, for example, a domi-nant or recessive allele. In one embodiment of the invention, a plant of lettuce line ADRIAN, MICKEY, BALBOA, or PAULIE is defined as comprising a single or multiple locus conversion. In specific embodiments of the invention, an added genetic locus confers one or more traits such as, for example, herbicide tolerance, insect resistance, disease resistance, and modified carbohydrate metabolism. In further embodiments, the trait may be conferred by a naturally occurring gene introduced into the genome of a line by backcrossing, a natural or induced mutation, or a transgene introduced through genetic transformation techniques into the plant or a progenitor of any previous generation thereof. When introduced through transformation, a genetic locus may comprise one or more genes integrated at a single chromo-somal location.

In some embodiments, a single locus conversion includes one or more site-specific changes to the plant genome, such as, without limitation, one or more nucleotide modifications, deletions, or insertions. A single locus may comprise one or more genes or nucleotides integrated or mutated at a single chromosomal location. In one embodiment, a single locus conversion may be introduced by a genetic engineering technique, methods of which include, for example, genome editing with engineered nucleases (GEEN). Engineered nucleases include, but are not limited to, Cas endonucleases; zinc finger nucleases (ZFNs); transcription activator-like effector nucleases (TALENs); engineered mega-nucleases, also known as homing endonucleases; and other endonucleases for DNA or RNA-guided genome editing that are well-known to the skilled artisan.

The invention further relates to methods for genetically modifying a lettuce plant that produces seed having resistance to Fusarium wilt, including but not limited to lettuce cultivar ADRIAN, MICKEY, BALBOA, or PAULIE, and to the modified lettuce plant produced by those methods. The genetic modification methods may include, but are not limited to mutation breeding, genome editing, RNA interference, gene silencing, backcross conversion, genetic transformation, single and multiple gene conversion, and/or direct gene transfer. The invention further relates to a genetically modified lettuce plant produced by the above methods, wherein the genetically modified lettuce plant comprises the genetic modification and otherwise comprises all of the physiological and morphological characteristics of a lettuce plant having resistance to Fusarium wilt, including but not limited to lettuce cultivar ADRIAN, MICKEY, BALBOA, or PAULIE.

The invention also concerns the seed of lettuce line ADRIAN, MICKEY, BALBOA, or PAULIE. The seed of the invention may be provided as an essentially homogeneous population of seed of lettuce line ADRIAN, MICKEY, BALBOA, or PAULIE. Essentially homogeneous populations of seed are generally free from substantial numbers of other seed. Therefore, the seed of lettuce line ADRIAN, MICKEY, BALBOA, or PAULIE may be defined as forming at least about 97% of the total seed, including at least 98%, 99%, or more of the seed. The seed population may be separately grown to provide an essentially homogeneous population of lettuce plants designated lettuce line ADRIAN, MICKEY, BALBOA, or PAULIE.

In yet another aspect, the invention relates to a tissue culture of regenerable cells of lettuce line ADRIAN, MICKEY, BALBOA, or PAULIE. The tissue culture will preferably be capable of regenerating lettuce plants capable of expressing all of the physiological and morphological characteristics of the starting plant and of regenerating plants having substantially the same genotype as the starting plant. Examples of some of the physiological and morphological characteristics of lettuce line ADRIAN, MICKEY, BALBOA, or PAULIE include those traits set forth in the tables herein. The regenerable cells in such tissue cultures may be derived, for example, from embryos, meristems, cotyledons, pollen, leaves, anthers, roots, root tips, pistils, flowers, seed, and stalks. Still further, the present invention provides lettuce plants regenerated from a tissue culture of the invention, the plants having all the physiological and morphological characteristics of lettuce line ADRIAN, MICKEY, BALBOA, or PAULIE.

In still yet another aspect of the invention, processes are provided for producing lettuce seeds, plants, and fruit, which processes generally comprise crossing a first parent lettuce plant with a second parent lettuce plant, wherein at least one of the first or second parent plants is a plant of lettuce line ADRIAN, MICKEY, BALBOA, or PAULIE. These processes may be further exemplified as processes for preparing hybrid lettuce seed or plants, wherein a first lettuce plant is crossed with a second lettuce plant of a different, distinct genotype to provide a hybrid that has, as one of its parents, a plant of lettuce line ADRIAN, MICKEY, BALBOA, or PAULIE. In these processes, crossing will result in the production of seed. The seed production occurs regardless of whether the seed is collected or not.

In one embodiment of the invention, the first step in “crossing” comprises planting seeds of a first and second parent lettuce plant, often in proximity so that pollination will occur for example, mediated by insect vectors. Alternatively, pollen can be transferred manually. Where the plant is self-pollinated, pollination may occur without the need for direct human intervention other than plant cultivation.

A second step may comprise cultivating or growing the seeds of first and second parent lettuce plants into plants that bear flowers. A third step may comprise preventing self-pollination of the plants, such as by emasculating the flowers (i.e. killing or removing the pollen).

A fourth step for a hybrid cross may comprise cross-pollination between the first and second parent lettuce plants. Yet another step comprises harvesting the seeds from at least one of the parent lettuce plants. The harvested seed can be grown to produce a lettuce plant or hybrid lettuce plant.

The present invention also provides the lettuce seeds and plants produced by a process that comprises crossing a first parent lettuce plant with a second parent lettuce plant, wherein at least one of the first or second parent lettuce plants is a plant of lettuce line ADRIAN, MICKEY, BALBOA, or PAULIE. In one embodiment of the invention, lettuce seed and plants produced by the process are first generation (F₁) hybrid lettuce seed and plants produced by crossing a plant in accordance with the invention with another, distinct plant. The present invention further contemplates plant parts of such an F₁ hybrid lettuce plant, and methods of use thereof. Therefore, certain exemplary embodiments of the invention provide an F₁ hybrid lettuce plant and seed thereof.

In still yet another aspect, the present invention provides a method of producing a plant derived from lettuce line ADRIAN, MICKEY, BALBOA, or PAULIE, the method comprising the steps of: (a) preparing a progeny plant derived from lettuce line ADRIAN, MICKEY, BALBOA, or PAULIE, wherein said preparing comprises crossing a plant of lettuce line ADRIAN, MICKEY, BALBOA, or PAULIE with a second plant; and (b) crossing the progeny plant with itself or a second plant to produce a seed of a progeny plant of a subsequent generation. In further embodiments, the method may additionally comprise: (c) growing a progeny plant of a subsequent generation from said seed of a progeny plant of a subsequent generation and crossing the progeny plant of a subsequent generation with itself or a second plant; and repeating the steps for an additional 3-10 generations to produce a plant derived from lettuce line ADRIAN, MICKEY, BALBOA, or PAULIE. The plant derived from lettuce line ADRIAN, MICKEY, BALBOA, or PAULIE may be an inbred line, and the aforementioned repeated crossing steps may be defined as comprising sufficient inbreeding to produce the inbred line. In the method, it may be desirable to select particular plants resulting from step (c) for continued crossing according to steps (b) and (c). By selecting plants having one or more desirable traits, a plant derived from lettuce line ADRIAN, MICKEY, BALBOA, or PAULIE is obtained which possesses some of the desirable traits of the line/hybrid as well as potentially other selected traits.

In certain embodiments, the present invention provides a method of producing food comprising: (a) obtaining a plant of lettuce line ADRIAN, MICKEY, BALBOA, or PAULIE, wherein the plant has been cultivated to maturity, and (b) collecting at least one lettuce from the plant.

In still yet another aspect of the invention, the genetic complement of lettuce line ADRIAN, MICKEY, BALBOA, or PAULIE is provided. The phrase “genetic complement” is used to refer to the aggregate of nucleotide sequences, the expression of which sequences defines the phenotype of, in the present case, a lettuce plant, or a cell or tissue of that plant. A genetic complement thus represents the genetic makeup of a cell, tissue or plant, and a hybrid genetic complement represents the genetic make-up of a hybrid cell, tissue or plant. The invention thus provides lettuce plant cells that have a genetic complement in accordance with the lettuce plant cells disclosed herein, and seeds and plants containing such cells.

Plant genetic complements may be assessed by genetic marker profiles, and by the expression of phenotypic traits that are characteristic of the expression of the genetic complement, e.g., isozyme typing profiles. It is understood that lettuce line ADRIAN, MICKEY, BALBOA, or PAULIE could be identified by any of the many well-known techniques such as, for example, Simple Sequence Length Polymorphisms (SSLPs) (Williams et al., Nucleic Acids Res., 1 8:6531-6535, 1990), Randomly Amplified Polymorphic DNAs (RAPDs), DNA Amplification Fingerprinting (DAF), Sequence Characterized Amplified Regions (SCARs), Arbitrary Primed Polymerase Chain Reaction (AP-PCR), Amplified Fragment Length Polymorphisms (AFLPs) (EP 534 858, specifically incorporated herein by reference in its entirety), and Single Nucleotide Polymorphisms (SNPs) (Wang et al., Science, 280:1077-1082, 1998).

In still yet another aspect, the present invention provides hybrid genetic complements, as represented by lettuce plant cells, tissues, plants, and seeds, formed by the combination of a haploid genetic complement of a lettuce plant of the invention with a haploid genetic complement of a second lettuce plant, preferably, another, distinct lettuce plant. In another aspect, the present invention provides a lettuce plant regenerated from a tissue culture that comprises a hybrid genetic complement of this invention.

Any embodiment discussed herein with respect to one aspect of the invention applies to other aspects of the invention as well, unless specifically noted.

Other objects, features, and advantages of the present invention will become apparent from the following detailed description. It should be understood, however, that the detailed description and any specific examples provided, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows a comparison of lettuce varieties HOT SHOT and BALBOA in fields located in south Yuma Valley, Arizona, in December 2022. Lettuce line BALBOA identifies Fusarium wilt tolerance.

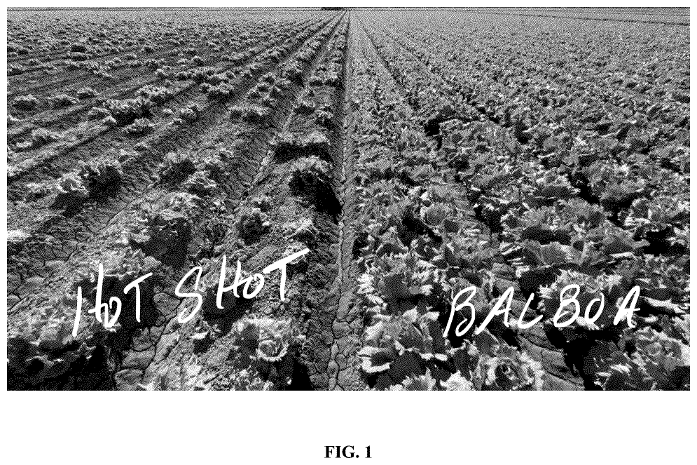

FIG. 2 shows a comparison of lettuce varieties ADRIAN and BIG SHOT in fields located in south Yuma Valley, Arizona, in December 2022. Lettuce line ADRIAN identifies Fusarium wilt tolerance.

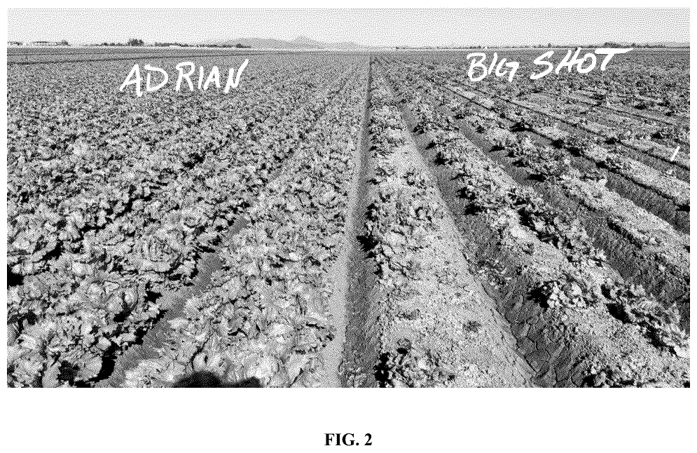

FIG. 3 shows QTLs identified for Fusarium resistance.

### DETAILED DESCRIPTION OF THE INVENTION

The present invention is directed to a lettuce line that produces plants and seeds with resistance to Fusarium wilt.

In the description and tables herein, a number of terms are used. In order to provide a clear and consistent understanding of the specification and claims, including the scope to be given such terms, the following definitions are provided:

The term “about” is used to indicate that a value includes the standard deviation of the mean for the device or method being employed to determine the value. The use of the term “or” in the claims is used to mean “and/or” unless explicitly indicated to refer to alternatives only or the alternatives are mutually exclusive. When used in conjunction with the word “comprising” or other open language in the claims, the words “a” and “an” denote “one or more,” unless specifi-cally noted otherwise. The terms “comprise,” “have,” and “include” are open-ended linking verbs. Any forms or tenses of one or more of these verbs, such as “comprises,” “com-prising,” “has,” “having,” “includes,” and “including,” are also open-ended. For example, any method that “comprises,” “has,” or “includes” one or more steps is not limited to possessing only those one or more steps and also covers other unlisted steps. Similarly, any plant that “comprises,” “has,” or “includes” one or more traits is not limited to possessing only those one or more traits and covers other unlisted traits.

Allele. An allele is any of one or more alternative forms of a gene which relate to one trait or characteristic. In a diploid cell or organism, the two alleles of a given gene occupy corresponding loci on a pair of homologous chromosomes.

Alter. The utilization of up-regulation, down-regulation, or gene silencing.

Backcrossing. Backcrossing is a process in which a breeder successively crosses hybrid progeny back to one of the parents, for example, a first-generation hybrid F₁ with one of the parental genotypes of the F₁ hybrid. Backcrossing can be used to introduce one or more single locus conversions or transgene from one genetic background.

Cell. Cell as used herein includes a plant cell, whether isolated, in tissue culture or incorporated in a plant or plant part.

Chlorosis. Used to describe a reduced amount of chlorophyll resulting in light or yellow-colored leaves.

Concentric chlorotic ring-spots. Light or dark areas on the leaf in the form concentric circles, ovals, or similar shape not necessarily symmetrical or uniform in appearance.

Cotyledon. A cotyledon is a type of seed leaf. The cotyledon contains the food storage tissues of the seed.

Crossing. The mating of two parent plants.

Cross-pollination: Fertilization by the union of two gametes from different plants.

Diploid: A cell or organism having two sets of chromosomes.

Emasculate: The removal of plant male sex organs or the inactivation of the organs with a cytoplasmic or nuclear genetic factor or a chemical agent conferring male sterility.

Embryo. The embryo is the small plant contained within a mature seed.

Enzyme. Enzymes are proteins that act as catalysts in all living organisms and serve as compounds that increase chemical reactions in biological systems.

Essentially all of the physiological and morphological characteristics. A plant having essentially all of the physiological and morphological characteristics means a plant having the physiological and morphological characteristics of the cultivar, except for the characteristics derived from the converted gene.

F₁ Hybrid. The first-generation progeny of the cross of two nonisogenic plants.

Gene expression. The process by which information encoded in a gene is used to direct the assembly of a functional product, such as a protein.

Gene silencing. The interruption or suppression of the expression of a gene at the level of transcription or translation.

Genetically modified. Describes an organism that has received genetic material from another organism, or had its genetic material modified, resulting in a change in one or more of its phenotypic characteristics. Methods used to modify, introduce, or delete the genetic material may include mutation breeding, genome editing, RNA interference, gene silencing, backcross conversion, genetic transformation, single and multiple gene conversion, and/or direct gene transfer.

Genome editing. A type of genetic engineering in which DNA is inserted, replaced, modified, or removed from a genome using artificially engineered nucleases or other targeted changes using homologous recombination. Examples include but are not limited to use of zinc finger nucleases (ZFNs), TAL effector nucleases (TALENs), meganucleases, CRISPR/Cas9, and other CRISPR related technologies. (Ma et. al., Molecular Plant, 9:961-974 (2016); Belhaj et. al., Current Opinion in Biotechnology, 32:76-84 (2015)).

Genotype: The genetic constitution of a cell or organism.

Haploid: A cell or organism having one set of the two sets of chromosomes in a diploid.

Hemizygous. Having or characterized by one or more genes that have no allelic counterparts. Hemizygous pertains to a diploid cell with only one copy of a gene instead of the usual two copies.

Hilum. This refers to the scar left on the seed which marks the place where the seed was attached to the pod prior to it (the seed) being harvested.

Leaflets. These are part of the plant shoot, and they manufacture food for the plant by the process of photosynthesis.

Leaf petiole. The small stalk attaching the leaf blade to the stem.

Leaf spots. A spot on a leaf usually resultant from infection; can be either chlorotic or necrotic and may be ringed, referred to as a ring-spot.

Linkage. Refers to a phenomenon wherein alleles on the same chromosome tend to segregate together more often than expected by chance if their transmission was independent.

Linkage disequilibrium. Refers to a phenomenon wherein alleles tend to remain together in linkage groups when segregating from parents to offspring, with a greater frequency than expected from their individual frequencies.

Locus. A locus confers one or more traits such as, for example, male sterility, insect resistance, disease resistance, and improved yield. The trait may be, for example, conferred by a naturally occurring gene introduced into the genome of the variety by backcrossing, a natural or induced mutation, or a transgene introduced through genetic transformation techniques. A locus may comprise one or more alleles integrated at a single chromosomal location.

Loss of function. As used herein, refers to a complete or partial loss of function or activity of an enzyme or protein.

M₁, M₂, M₃, etc. Used to indicate the generations after a mutational treatment, analogous to filial generation F₁, F₂, etc. that identifies generations after a hybridization of two individuals that are advanced through self-fertilization.

Marker. A readily detectable phenotype, preferably inherited in codominant fashion (both alleles at a locus in a diploid heterozygote are readily detectable), with no environmental variance component, i.e., heritability of 1.

Maturity Date. Plants are considered mature when about 70% of the pods display a dark brown or black endocarp on the middle upper side of the pod. The endocarp is revealed by removing the exocarp by mechanical scraping or by pressurized water.

Modulating or modulation of expression of a gene. As used herein, refers to changes or alterations to a gene produced by techniques including but not limited to gene suppression, gene down-regulation, gene knock-down, gene mutation, gene silencing, gene (genome) editing, and other techniques that result in a change in the expression of a gene as compared to a wild-type gene.

Mottling. Abnormal coloration on plants, usually a sign of disease or malnutrition.

Multiple Gene Converted (Conversion). Multiple gene converted (conversion) includes plants developed by a plant breeding technique called backcrossing wherein essentially all of the desired morphological and physiological characteristics of a variety are recovered, while retaining two or more genes transferred into the variety via crossing and backcrossing. The term can also refer to the introduction of multiple genes through genetic engineering techniques known in the art.

Non-natural mutation(s). Refers to one or more mutation(s) in a plant that does not occur naturally. Non-natural mutations occur with artificial external intervention, as opposed to spontaneous or hereditary mutations.

Pedigree Distance. Relationship among generations based on their ancestral links as evidenced in pedigrees. May be measured by the distance of the pedigree from a given starting point in the ancestry.

Plant. As used herein, the term “plant” includes reference to an immature or mature whole plant, including a plant from which seed or grain or anthers have been removed. A seed or embryo that will produce the plant is also considered to be the plant.

Plant Parts. As used herein, the term “plant parts” (or a part thereof) includes protoplasts, leaves, stems, roots, root tips, anthers, pistils, seed, grain, embryo, pollen, ovules, cotyledon, hypocotyl, panicles, flower, shoot, tissue, petiole, cells, meristematic cells and the like.

Plastids. Small, double-membraned organelles of plant cells that contain their own DNA and ribosomes. Some plastids, such as the chloroplasts in plant leaves, contain pigments used in photosynthesis.

Quantitative Trait Loci (QTL). Refers to genetic loci that control to some degree numerically representable traits that are usually continuously distributed.

Regeneration: The development of a plant from tissue culture.

Resistance. As used herein, the terms “resistance” and “tolerance” are used interchangeably to describe plants that show no symptoms to a specified biotic pest, pathogen, abiotic influence, or environmental condition. These terms are also used to describe plants showing some symptoms but that are still able to produce marketable products with an acceptable yield. Some plants that are referred to as resistant or tolerant are only so in the sense that they may still produce a crop, even though the plants are stunted, and the yield is reduced.

Self-pollination. The transfer of pollen from the anther to the stigma of the same plant.

Single Gene Converted (Conversion). Single gene converted (conversion) plant refers to plants which are developed by a plant breeding technique called backcrossing with selection wherein essentially all of the desired morphological and physiological characteristics of a variety are recovered in addition to the single gene transferred into the variety via the backcrossing technique or via genetic engineering.

Single Locus Converted (Conversion) Plant. Plants which are developed by a plant breeding technique called backcrossing or genetic engineering of a locus, wherein essentially all of the morphological and physiological characteristics of a lettuce variety are recovered in addition to the characteristics of the single locus.

Substantially equivalent. A characteristic that, when compared, does not show a statistically significant difference (e.g., p=0.05) from the mean.

Tissue Culture. A composition comprising isolated cells of the same or a different type or a collection of such cells organized into parts of a plant.

Plant Transgenic. A genetic locus comprising a sequence which has been introduced into the genome of a lettuce plant by transformation or site-specific modification.

The invention provides methods and compositions relating to plants, seeds, and derivatives of lettuce line ADRIAN, MICKEY, BALBOA, or PAULIE. Lettuce line ADRIAN, MICKEY, BALBOA, and PAULIE of the present invention are resistant to Fusarium wilt. Lettuce line ADRIAN, MICKEY, BALBOA, and PAULIE of the present invention have the following morphologic and other characteristics based on data collected in Yuma, Arizona.

### Example 1

### Fusarium Wilt Resistant Variety Adrian

ADRIAN was developed from a cross between Apollo Creed×NUN 00236 using the pedigree breeding method and was released at the F₆ level.

### Plant:

Type: Iceberg lettuce
Maturity date: Dec. 29, 2022

### Seed:

Color: White
Light dormancy: None
Heat dormancy: None

### Cotyledon (to Fourth Leaf Stage):

Green color: Green
Anthocyanin distribution: None/absent

### Mature Leaves:

Margin: Moderate
   Incision depth: Shallow
   Indentation: Crenate
      Undulation of the apical margin: Moderate
      Green color (at harvest maturity): Green
      Hue of green color of outer leaves: Semi glossy
      Intensity of color of outer leaves: Medium
   Anthocyanin distribution: Absent

### Plant (at Market Stage):

Spread of frame leaves: 50.8 cm
Head diameter (cm): 15.7 cm
Head shape: Round to pointed round
Head size class: Medium large
Head weight (g): 626.0 g
Head firmness: Semi firm

### Butt:

Shape: Flat round
Midrib: Medium raised

### Core:

Diameter at base of head: 3.1 cm
Core height from base of head to apex: 2.7 cm

### Bolting:

First water date: Sep. 28, 2022
Number of days from first water date to seed stalk emergence: 124 days
Time of beginning of bolting: Late
Class: Late
Height of mature seed stalk: 76.3 cm
Spread of bolter plant: 31.3 cm
Bolter leaves: Broad
Bolter leaf margin: Crenate
Bolter leaf color: Green
Bolter habit:
   Terminal inflorescence: Present
   Lateral shoots: Present
   Basal side shoots: Absent

### Primary Regions of Adaptation:

Autumn area: Yuma, Arizona
Winter area: Yuma, Arizona

### Disease/Pest Resistance:

Fusarium oxysporum f. sp. lactucae: High tolerance

### Physiological Responses

Tipburn: Tolerant

### Example 2

### Fusarium Wilt Resistant Variety Mickey

MICKEY was developed from a cross between Apollo Creed×NUN 00236 using the pedigree breeding method and was released at the F₆ level.

### Plant:

Type: Iceberg lettuce
Maturity date: Dec. 29, 2022

### Seed:

Color: White
Light dormancy: No
Heat dormancy: No

### Mature Leaves:

Margin:
   Incision depth: Shallow
   Indentation: Crenate
   Undulation of the apical margin: Slight
   Green color (at harvest maturity): Green
   Hue of green color of outer leaves: Slightly glossy
   Intensity of color of outer leaves: Medium
Anthocyanin distribution: Absent

### Plant (at Market Stage):

Spread of frame leaves: 53.7 cm
Head diameter (cm): 15.4 cm
Head shape: Flat round
Head size class: Medium large
Head weight (g): 706.3 g
Head firmness: Slightly firm

### Butt:

Shape: Flat round
Midrib: Slightly protruding

### Core:

Diameter at base of head: 3.8 cm
Core height from base of head to apex: 3.4 cm

### Bolting:

First water date: Sep. 29, 2022
Number of days from first water date to seed stalk emergence: 110 days
Time of beginning of bolting: Late
Class: Slow bolting
Height of mature seed stalk: 109.0 cm
Spread of bolter plant: 29.9 cm
Bolter leaves: Broad
Bolter leaf margin: Crenate
Bolter leaf color: Green
Bolter habit:
   Terminal inflorescence: Present
   Lateral shoots: Present
   Basal side shoots: Absent

### Primary Regions of Adaptation:

Autumn area: Yuma, Arizona
Winter area: Yuma, Arizona

### Disease/Pest Resistance:

Fusarium oxysporum f. sp. lactucae: High tolerance

### Physiological Responses:

Tipburn: Tolerant

### Example 3

### Fusarium Wilt Resistant Variety Balboa

BALBOA was developed from a cross between Apollo Creed×NUN 00236 using the pedigree breeding method and was released at the F₆ level.

### Plant:

Type: Iceberg lettuce
Maturity date: Dec. 29, 2022

### Seed:

Light dormancy: No
Heat dormancy: No

### Mature Leaves:

Margin:
   Incision depth: Shallow
   Indentation: Crenate
   Undulation of the apical margin: Slight
   Green color (at harvest maturity): Green
   Hue of green color of outer leaves: Slightly glossy
   Intensity of color of outer leaves: Medium
Anthocyanin distribution: Absent

### Plant (at Market Stage):

Spread of frame leaves: 51.1 cm
Head diameter (cm): 15.5 cm
Head shape: Flat round
Head size class: Medium large
Head weight (g): 625.5 g
Head firmness: Semi firm

### Butt:

Shape: Flat round
Midrib: Moderately raised

### Core:

Diameter at base of head: 3.6 cm
Core height from base of head to apex: 2.6 cm

### Bolting:

First water date: Sep. 28, 2022
Number of days from first water date to seed stalk emergence: 117 days
Time of beginning of bolting: Late
Class: Slow
Height of mature seed stalk: 93.0 cm
Spread of bolter plant: 27.5 cm
Bolter leaves: Broad
Bolter leaf margin: Crenate
Bolter leaf color: Green
Bolter habit:
   Terminal inflorescence: Present
   Lateral shoots: Present
   Basal side shoots: Absent

### Primary Regions of Adaptation:

Autumn area: Yuma, Arizona
Winter area: Yuma, Arizona

### Disease/Pest Resistance:

Fusarium oxysporum f. sp. lactucae: High tolerance

### Physiological Responses:

Tipburn: Tolerant

### Example 4

### Fusarium Wilt Resistant Variety Paulie

PAULIE was developed from a cross between Apollo Creed×NUN 00236 using the pedigree breeding method and was released at the F₆ level.

### Plant:

Type: Iceberg lettuce
Maturity date: Dec. 29, 2022

### Seed:

Color: White
Light dormancy: None
Heat dormancy: None

### Cotyledon (to Fourth Leaf Stage):

Green color: Green
Anthocyanin distribution: None/absent

### Mature Leaves:

Margin: Moderate
Incision depth: Shallow
Indentation: Crenate
Undulation of the apical margin: Moderate
Green color (at harvest maturity): Green
Hue of green color of outer leaves: Semi glossy
Intensity of color of outer leaves: Medium
Anthocyanin distribution: Absent
   Plant (at market stage):
Spread of frame leaves: 53.2 cm
Head diameter (cm): 14.58 cm
Head shape: Flat round
Head size class: Medium large
Head weight (g): 573.0 g
Head firmness: Semi firm

### Butt:

Shape: Flat round
Midrib: Medium raised

### Core:

Diameter at base of head: 3.4 cm
Core height from base of head to apex: 2.3 cm

### Bolting:

First water date: Sep. 28, 2022
Number of days from first water date to seed stalk emergence: 124 days
Time of beginning of bolting: Late
Class: Late
Height of mature seed stalk: 79.3 cm
Spread of bolter plant: 53.19 cm
Bolter leaves: Broad
Bolter leaf margin: Crenate
Bolter leaf color: Green
Bolter habit:
   Terminal inflorescence: Present
   Lateral shoots: Present
   Basal side shoots: Absent

### Primary Regions of Adaptation:

Autumn area: Yuma, Arizona
Winter area: Yuma, Arizona

### Disease/Pest Resistance:

Fusarium oxysporum f. sp. lactucae: High tolerance

### Physiological Responses:

Tipburn: Tolerant

Trichome types were evaluated based on the categories described by Luckwill, L. (The genus Lycopersicon: historical, biological, and taxonomic survey of the wild and cultivated lettuce; Aberdeen University Press, Aberdeen, Scotland, 1943) and Glas, et al. (Plant Glandular Trichomes as Targets for Breeding or Engineering of Resistance to Herbivores. Int. J. Mol. Sci. 2012, 13, 17077-17103; doi:10.3390/ijms131217077).

### Example 5

### Identification of QTLS Linked to Fusarium Ewsistance

One hundred and forty F₂ individuals derived from one biparental population namely NUN 236×NUN 1204 was used to identify the Fusarium resistance QTL. The parents used in generating the biparental populations were also included/Phenotypic data for the F₂ individuals and the parents were used in the investigations.

DNA was extracted from all the individuals from lyophilized leaf tissues submitted by the customer using a proprietary silica-based extraction chemistry. DNA concentration was quantified using Biotium Green. Quality/integrity of the DNA was assessed by both the A260/A280 ratio using a Nanodrop and gel electrophoresis. All the assessments were carried out according to the instructions of the manufacturer.

DNA was normalized to 20 ng/μL and used for generating the Restriction site associated DNA sequencing (RADSeq) libraries, which were subsequently sequenced on a NovaSeq 6000 platform using a S1 flow cell to generate 150 bp paired end reads.

Demultiplexed FastQ reads were assessed for initial QC and then subjected to variant/Single Nucleotide Polymorphism (SNP) analysis to identify polymorphic markers. Markers were obtained by aligning the reads against a lettuce reference genome, (Lactuca sativa) ‘Salinas’ strain https colon slash slash www period ncbi period nlm period nih.gov/datasets/genome/GCA_0028700754/). For read alignment and SNP calling, default parameters were used in Burrows-Wheeler Aligner, and Samtools/Bcftools, respectively. SNPs obtained were filtered to retain those with: Minimum average read depth of 10; Minimum quality score (Q) of 30; Minor allele frequency of 3% and Maximum missing allelic data of 10%. Markers that had a missing genotype for one of the two parents were not used in the creation of linkage maps and hence the QTL mapping.

Polymorphic and unique marker sets were used. The markers were ‘binned’ to remove highly similar markers and were then grouped, ordered, and rippled to create linkage maps using default parameters in IciMapping 4.2.53.

QTL mapping was done using the inclusive composite interval mapping (ICIM) model in IciMapping 4.2.53. LOD threshold of 7.0 was used as trait permutations could not resolve a recommended threshold level in this case.

Sequences encompassing the SNPs were extracted from all the markers corresponding to the respective QTLs and were used to design KASP markers (Table 1). All the extracted DNA were genotyped using KASP specific PCR cycling conditions and validated on the ATP platform using a proprietary reaction set up.

TABLE 1
Primer Sequences with SNP and Sequence Information
Used for Evaluating Fusarium Resistance in Lettuce

| | | Primer | | |
|---|---|---|---|---|
| | KASP ID | Sequence | | |
| Original | For | (SEQ | | Sequence |
| SNP ID | Evaluation | ID NO) | SNP | with SNP |
| CM022523.2_{—} | FusRes 6R | 1 | G/A | 5 |
| 16507009 | | 2 | | |
| | | 3 | | |
| | | 4 | | |
| CM022523.2_{—} | FusRes 6L | 6 | A/C | 10 |
| 51808919 | | 7 | | |
| | | 8 | | |
| | | 9 | | |
| CM022526.2_{—} | FusRes 9R | 11 | A/G | 15 |
| 165016132 | | 12 | | |
| | | 13 | | |
| | | 14 | | |
| CM022526.2_{—} | FusRes 9L | 16 | T/A | 20 |
| 211881415 | | 17 | | |
| | | 18 | | |
| | | 19 | | |
| CM022522.2_{—} | FusRes 5R | 21 | G/A | 25 |
| 75452593 | | 22 | | |
| | | 23 | | |
| | | 24 | | |
| CM022522.2_{—} | FusRes 5L | 26 | A/T | 30 |
| 128810019 | | 27 | | |
| | | 28 | | |
| | | 29 | | |
| CM022519.2_{—} | FusRes 2R | 31 | T/C | 35 |
| 10384499 | | 32 | | |
| | | 33 | | |
| | | 34 | | |
| CM022519.2_{—} | FusRes 2L | 36 | C/G | 40 |
| 10384545 | | 37 | | |
| | | 38 | | |
| | | 39 | | |

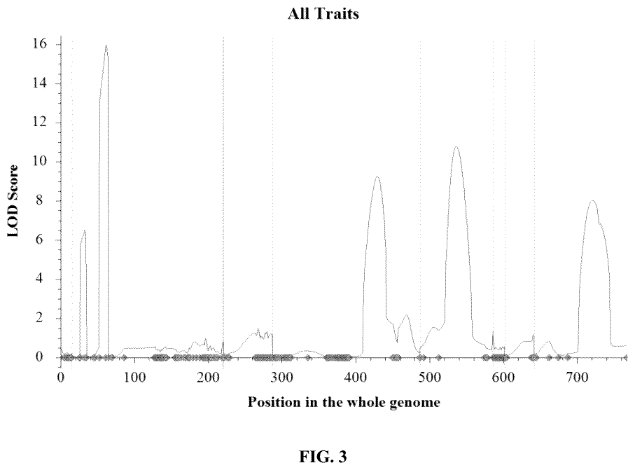

No significant QTL/s were identified for Fusarium resistance from the Nun 236×Nun 1204 population using standard trait permutation threshold levels. However, 76 QTLs were identified after the LOD threshold was set to 7.0, across several chromosomes ((FIG. 3). Four candidates from chromosomes 2, 5, 6 and 9 were selected for further analysis. The original SNP identifications, KASP IDs, primer sequences, SNPs and sequences with the SNPs are set forth in Table 1. The sequences set forth by SEQ ID NOS. refer to the SEQ ID NOs set forth in the Sequence Listing. Further markers can be derived from the SNP sequences using conventional techniques.

### Breeding Lettuce Plants

The present invention is directed to a lettuce line that produces plants and seeds with resistance to Fusarium wilt.

One aspect of the current invention concerns methods for producing seed of lettuce line ADRIAN, MICKEY, BALBOA, or PAULIE. Alternatively, in other embodiments of the invention, lettuce line ADRIAN, MICKEY, BALBOA, or PAULIE may be crossed with itself or with any second plant. Such methods can be used for propagation of lettuce line ADRIAN, MICKEY, BALBOA, or PAULIE or can be used to produce plants that are derived from ADRIAN, MICKEY, BALBOA, or PAULIE. Plants derived from lettuce line ADRIAN, MICKEY, BALBOA, or PAULIE may be used, in certain embodiments, for the development of new lettuce varieties.

The development of new varieties using one or more starting varieties is well-known in the art. In accordance with the invention, novel varieties may be created by crossing lettuce line ADRIAN, MICKEY, BALBOA, or PAULIE followed by multiple generations of breeding according to such well-known methods.

New varieties may be created by crossing with any second plant. In selecting such a second plant to cross for the purpose of developing novel lines, it may be desired to choose those plants which either themselves exhibit one or more selected desirable characteristics or which exhibit the desired characteristic(s) when in hybrid combination. Once initial crosses have been made, inbreeding and selection take place to produce new varieties. For development of a uniform line, often five or more generations of selfing and selection are involved.

Uniform lines of new varieties may also be developed by way of double-haploids. This technique allows the creation of true breeding lines without the need for multiple genera-tions of selfing and selection. In this manner true breeding lines can be produced in as little as one generation. Haploid embryos may be produced from microspores, pollen, anther cultures, or ovary cultures. The haploid embryos may then be doubled autonomously, or by chemical treatments (e.g., colchicine treatment). Alternatively, haploid embryos may be grown into haploid plants and treated to induce chromosome doubling. In either case, fertile homozygous plants are obtained. In accordance with the invention, any of such techniques may be used in connection with a plant of the invention and progeny thereof to achieve a homozygous line.

Backcrossing can also be used to improve an inbred plant. Backcrossing transfers a specific desirable trait from one inbred or non-inbred source to an inbred that lacks that trait. This can be accomplished, for example, by first crossing a superior inbred (A) (recurrent parent) to a donor inbred (non-recurrent parent), which carries the appropriate locus or loci for the trait in question. The progeny of this cross are then mated back to the superior recurrent parent (A) followed by selection in the resultant progeny for the desired trait to be transferred from the non-recurrent parent. After five or more backcross generations with selection for the desired trait, the progeny have the characteristic being transferred but are like the superior parent for most or almost all other loci. The last backcross generation would be selfed to give pure breeding progeny for the trait being transferred.

The plants of the present invention are particularly well suited for the development of new lines based on the elite nature of the genetic background of the plants. In selecting a second plant to cross with lettuce line ADRIAN, MICKEY, BALBOA, or PAULIE for the purpose of developing novel lettuce lines, it will typically be preferred to choose those plants which either themselves exhibit one or more selected desirable characteristics or which exhibit the desired characteristic(s) when in hybrid combination. Examples of desirable traits may include, in specific embodiments, high seed yield, high seed germination, seed-ling vigor, high fruit yield, disease tolerance or resistance, and adaptability for soil and climate conditions. Consumer driven traits, such as a fruit shape, color, texture, and taste are other examples of traits that may be incorporated into new lines of lettuce plants developed by this invention.

In certain aspects of the invention, plants described herein are provided modified to include at least a first desired heritable trait. Such plants may, in one embodiment, be developed by a plant breeding technique called backcrossing, wherein essentially all of the morphological and physi-ological characteristics of a variety are recovered in addition to a genetic locus transferred into the plant via the backcrossing technique. The term single locus converted plant as used herein refers to those lettuce plants which are developed by a plant breeding technique called backcrossing or by genetic engineering, wherein essentially all of the morphological and physiological characteristics of a variety are recovered or conserved in addition to the single locus introduced into the variety via the backcrossing or genetic engineering technique, respectively. By essentially all of the morphological and physiological characteristics, it is meant that the characteristics of a plant are recovered or conserved that are otherwise present when compared in the same environment, other than an occasional variant trait that might arise during backcrossing, introduction of a transgene, or application of a genetic engineering technique.

Backcrossing methods can be used with the present invention to improve or introduce a characteristic into the present variety. The parental lettuce plant which contributes the locus for the desired characteristic is termed the nonre-current or donor parent. This terminology refers to the fact that the nonrecurrent parent is used one time in the backcross protocol and therefore does not recur. The parental lettuce plant to which the locus or loci from the nonrecurrent parent are transferred is known as the recurrent parent as it is used for several rounds in the backcrossing protocol.

In a typical backcross protocol, the original variety of interest (recurrent parent) is crossed to a second variety (nonrecurrent parent) that carries the single locus of interest to be transferred. The resulting progeny from this cross are then crossed again to the recurrent parent and the process is repeated until a lettuce plant is obtained wherein essentially all of the morphological and physiological characteristics of the recurrent parent are recovered in the converted plant, in addition to the single transferred locus from the nonrecurrent parent.

The selection of a suitable recurrent parent is an important step for a successful backcrossing procedure. The goal of a backcross protocol is to alter or substitute a single trait or characteristic in the original variety. To accomplish this, a single locus of the recurrent variety is modified or substi-tuted with the desired locus from the nonrecurrent parent, while retaining essentially all of the rest of the desired genetic, and therefore the desired physiological and mor-phological constitution of the original variety. The choice of the particular non-recurrent parent will depend on the purpose of the backcross; one of the major purposes is to add some commercially desirable trait to the plant. The exact backcrossing protocol will depend on the characteristic or trait being altered and the genetic distance between the recurrent and nonrecurrent parents. Although backcrossing methods are simplified when the characteristic being transferred is a dominant allele, a recessive allele, or an additive allele (between recessive and dominant), may also be transferred. In this instance it may be necessary to introduce a test of the progeny to determine if the desired characteristic has been successfully transferred.

In one embodiment, progeny lettuce plants of a backcross in which a plant described herein is the recurrent parent comprise (i) the desired trait from the non-recurrent parent and (ii) all of the physiological and morphological charac-teristics of lettuce the recurrent parent as determined at the 5% significance level when grown in the same environmental conditions.

New varieties can also be developed from more than two parents. The technique, known as modified backcrossing, uses different recurrent parents during the backcrossing. Modified backcrossing may be used to replace the original recurrent parent with a variety having certain more desirable characteristics or multiple parents may be used to obtain different desirable characteristics from each.

With the development of molecular markers associated with particular traits, it is possible to add additional traits into an established germ line, such as represented here, with the end result being substantially the same base germplasm with the addition of a new trait or traits. Molecular breeding, as described in Moose and Mumm, 2008 (Plant Physiol., 147: 969-977), for example, and elsewhere, provides a mechanism for integrating single or multiple traits or QTL into an elite line. This molecular breeding-facilitated movement of a trait or traits into an elite line may encompass incorporation of a particular genomic fragment associated with a particular trait of interest into the elite line by the mechanism of identification of the integrated genomic frag-ment with the use of flanking or associated marker assays. In the embodiment represented here, one, two, three or four genomic loci, for example, may be integrated into an elite line via this methodology. When this elite line containing the additional loci is further crossed with another parental elite line to produce hybrid offspring, it is possible to then incorporate at least eight separate additional loci into the hybrid. These additional loci may confer, for example, such traits as a disease resistance or a fruit quality trait. In one embodiment, each locus may confer a separate trait. In another embodiment, loci may need to be homozygous and exist in each parent line to confer a trait in the hybrid. In yet another embodiment, multiple loci may be combined to confer a single robust phenotype of a desired trait.

Many single locus traits have been identified that are not regularly selected in the development of a new inbred but that can be improved by backcrossing techniques. Single locus traits may or may not be transgenic; examples of these traits include, but are not limited to, herbicide resistance, resistance to bacterial, fungal, or viral disease, insect resis-tance, modified fatty acid or carbohydrate metabolism, and altered nutritional quality. These comprise genes generally inherited through the nucleus.

Direct selection may be applied where the single locus acts as a dominant trait. For this selection process, the progeny of the initial cross is assayed for viral resistance or the presence of the corresponding gene prior to the back-crossing. Selection eliminates any plants that do not have the desired gene and resistance trait, and only those plants that have the trait are used in the subsequent backcross. This process is then repeated for all additional backcross generations.

Selection of lettuce plants for breeding is not necessarily dependent on the phenotype of a plant and instead can be based on genetic investigations. For example, one can utilize a suitable genetic marker which is closely genetically linked to a trait of interest. One of these markers can be used to identify the presence or absence of a trait in the offspring of a particular cross and can be used in selection of progeny for continued breeding. This technique is commonly referred to as marker assisted selection. Any other type of genetic marker or other assay which is able to identify the relative presence or absence of a trait of interest in a plant can also be useful for breeding purposes. Procedures for marker assisted selection are well known in the art. Such methods will be of particular utility in the case of recessive traits and variable phenotypes, or where conventional assays may be more expensive, time consuming, or otherwise disadvantageous. In addition, marker assisted selection may be used to identify plants comprising desirable genotypes at the seed, seedling, or plant stage, to identify or assess the purity of a cultivar, to catalog the genetic diversity of a germplasm collection, and to monitor specific alleles or haplotypes within an established cultivar.

Types of genetic markers which could be used in accordance with the invention include, but are not necessarily limited to, Simple Sequence Length Polymorphisms (SSLPs) (Williams et al., Nucleic Acids Res., 1 8:6531-6535, 1990), Randomly Amplified Polymorphic DNAs (RAPDs), DNA Amplification Fingerprinting (DAF), Sequence Char-acterized Amplified Regions (SCARs), Arbitrary Primed Polymerase Chain Reaction (AP-PCR), Amplified Fragment Length Polymorphisms (AFLPs) (EP 534 858, specifically incorporated herein by reference in its entirety), and Single Nucleotide Polymorphisms (SNPs) (Wang et al., Science, 280:1077-1082, 1998).

In particular embodiments of the invention, marker assisted selection is used to increase the efficiency of a backcrossing breeding scheme for producing a lettuce line comprising a desired trait. This technique is commonly referred to as marker assisted backcrossing (MABC). This technique is well-known in the art and may involve, for example, the use of three or more levels of selection, including foreground selection to identity the presence of a desired locus, which may complement or replace phenotype screening protocols; recombinant selection to minimize linkage drag; and background selection to maximize recur-rent parent genome recovery.

### Plants Derived by Genetic Engineering

Various genetic engineering technologies have been developed and may be used by those of skill in the art to introduce traits in plants. In certain aspects of the claimed invention, traits are introduced into lettuce plants via alter-ing or introducing a single genetic locus or trans gene into the genome of a recited variety or progenitor thereof. Methods of genetic engineering to modify, delete, or insert genes and polynucleotides into the genomic DNA of plants are well known in the art.

In specific embodiments of the invention, improved lettuce lines can be created through the site-specific modi-fication of a plant genome. Methods of genetic engineering include, for example, utilizing sequence-specific nucleases such as zinc-finger nucleases (see, for example, U.S. Pat. Appl. Pub. No. 2011-0203012); engineered or native mega-nucleases; TALE-endonucleases (see, for example, U.S. Pat. Nos. 8,586,363 and 9,181,535); and RNA-guided endonu-cleases, such as those of the CRISPR/Cas systems (see, for example, U.S. Pat. Nos. 8,697,359 and 8,771,945 and U.S. Pat. Appl. Pub. No. 2014-0068797). One embodiment of the invention thus relates to utilizing a nuclease or any associ-ated protein to carry out genome modification. This nuclease could be provided heterologously within donor template DNA for templated-genomic editing or in a separate molecule or vector. A recombinant DNA construct may also comprise a sequence encoding one or more guide RNAs to direct the nuclease to the site within the plant genome to be modified. Further methods for altering or introducing a single genetic locus include, for example, utilizing single-stranded oligonucleotides to introduce base pair modifica-tions in a lettuce plant genome (see, for example Sauer et al., Plant Physiol, 170(4):1917-1928, 2016).

Methods for site-directed alteration or introduction of a single genetic locus are well-known in the art and include those that utilize sequence-specific nucleases, such as the aforementioned, or complexes of proteins and guide-RNA that cut genomic DNA to produce a double-strand break (DSB) or nick at a genetic locus. As is well-understood in the art, during the process of repairing the DSB or nick intro-duced by the nuclease enzyme, a donor template, transgene, or expression cassette polynucleotide may become inte-grated into the genome at the site of the DSB or nick. The presence of homology arms in the DNA to be integrated may promote the adoption and targeting of the insertion sequence 30 into the plant genome during the repair process through homologous recombination or non-homologous end joining (NHEJ).

In another embodiment of the invention, genetic transformation may be used to insert a selected transgene into a plant of the invention or may, alternatively, be used for the preparation of transgenes which can be introduced by back-crossing. Methods for the transformation of plants that are well-known to those of skill in the art and applicable to many crop species include, but are not limited to, electropo-ration, microprojectile bombardment, Agrobacterium-medi-ated transformation, and direct DNA uptake by protoplasts.

To effect transformation by electroporation, one may employ either friable tissues, such as a suspension culture of cells or embryogenic callus or alternatively one may trans-form immature embryos or other organized tissue directly. In this technique, one would partially degrade the cell walls of the chosen cells by exposing them to pectin-degrading enzymes (pectolyases) or mechanically wound tissues in a controlled manner.

An efficient method for delivering transforming DNA segments to plant cells is microprojectile bombardment. In this method, particles are coated with nucleic acids and delivered into cells by a propelling force. Exemplary particles include those comprised of tungsten, platinum, and preferably, gold. For the bombardment, cells in suspension are concentrated on filters or solid culture medium. Alter-natively, immature embryos or other target cells may be arranged on solid culture medium. The cells to be bom-barded are positioned at an appropriate distance below the macroprojectile stopping plate.

An illustrative embodiment of a method for delivering DNA into plant cells by acceleration is the Biolistics Particle Delivery System, which can be used to propel particles coated with DNA or cells through a screen, such as a stainless steel or Nytex screen, onto a surface covered with target cells. The screen disperses the particles so that they are not delivered to the recipient cells in large aggregates. Microprojectile bombardment techniques are widely applicable and may be used to transform virtually any plant species.

Agrobacterium-mediated transfer is another widely appli-cable system for introducing gene loci into plant cells. An advantage of the technique is that DNA can be introduced into whole plant tissues, thereby bypassing the need for regeneration of an intact plant from a protoplast. Modem Agrobacterium transformation vectors are capable of replication in E. coli as well as Agrobacterium, allowing for convenient manipulations (Klee et al., Nat. Biotechnol., 3(7):637-642, 1985). Moreover, recent technological advances in vectors for Agrobacterium-mediated gene trans-fer have improved the arrangement of genes and restriction sites in the vectors to facilitate the construction of vectors capable of expressing various polypeptide coding genes. The vectors described have convenient multi-linker regions flanked by a promoter and a polyadenylation site for direct expression of inserted polypeptide coding genes. Additionally, Agrobacterium containing both armed and disarmed Ti genes can be used for transformation.

In those plant strains where Agrobacterium-mediated transformation is efficient, it is the method of choice because of the facile and defined nature of the gene locus transfer. The use of Agrobacterium-mediated plant integrating vec-tors to introduce DNA into plant cells is well known in the art (Fraley et al., Nat. Biotechnol., 3:629-635, 1985; U.S. Pat. No. 5,563,055).

Transformation of plant protoplasts also can be achieved using methods based on calcium phosphate precipitation, polyethylene glycol treatment, electroporation, and combi-nations of these treatments (see, for example, Potrykus et al., Mal. Gen. Genet., 199:183-188, 1985; Omirulleh et al., Plant Mal. Biol., 21(3):415-428, 1993; Fromm et al., Nature, 312:791-793, 1986; Uchimiya et al., Mal. Gen. Genet., 204:204, 1986; Marcotte et al., Nature, 335:454, 1988). Transformation of plants and expression of foreign genetic elements is exemplified in Choi et al. (Plant Cell Rep., 13:344-348, 1994), and Ellul et al. (Theor. Appl. Genet., 1 07:462-469, 2003).

A number of promoters have utility for plant gene expres-sion for any gene of interest including but not limited to selectable markers, scoreable markers, genes for pest tolerance, disease resistance, nutritional enhancements and any other gene of agronomic interest. Examples of constitutive promoters useful for plant gene expression include, but are not limited to, the cauliflower mosaic virus (CaMV) P-35S promoter, which confers constitutive, high-level expression in most plant tissues (see, for example, Odel et al., Nature, 313:810, 1985), including in monocots (see, for example, Dekeyser et al., Plant Cell, 2:591, 1990; Terada and Shi-mamoto, Mal. Gen. Genet., 220:389, 1990); a tandemly duplicated version of the CaMV 35S promoter, the enhanced 35S promoter (P-e35S); the nopaline synthase promoter (An et al., Plant Physiol., 88:547, 1988); the octopine synthase promoter (Fromm et al., Plant Cell, 1:977, 1989); the figwort mosaic virus (P-FMV) promoter as described in U.S. Pat. No. 5,378,619; an enhanced version of the FMV promoter (P-eFMV) where the promoter sequence of P-FMV is duplicated in tandem; the cauliflower mosaic virus 19S promoter; a sugarcane bacilliform virus promoter; a commelina yellow mottle virus promoter; and other plant virus promoters known to express in plant cells.

A variety of plant gene promoters that are regulated in response to environmental, hormonal, chemical, or developmental signals can also be used for expression of an operably linked gene in plant cells, including promoters regulated by (1) heat (Callis et al., Plant Physiol., 88:965, 1988), (2) light (e.g., pea rbcS-3A promoter, Kuhlemeier et al., Plant Cell, 1:471, 1989; maize rbcS promoter, Schaffner and Sheen, Plant Cell, 3:997, 1991; or chlorophyll alb—5 binding protein promoter, Simpson et al., EMBO J., 4:2723, 1985), (3) hormones, such as abscisic acid (Marcotte et al., Plant Cell, 1:969, 1989), (4) wounding (e.g., wunl, Siebertz et al., Plant Cell, 1:961, 1989); or (5) chemicals, such as methyl jasmonate, salicylic acid, or Safener. It may also be 10 advantageous to employ organ-specific promoters (e.g., Roshal et al., EMBO J., 6:1155, 1987; Schernthaner et al., EMBO J., 7:1249, 1988; Bustos et al., Plant Cell, 1:839, 1989).

Exemplary nucleic acids which may be introduced to plants of this invention include, for example, DNA sequences or genes from another species, or even genes or sequences which originate with or are present in the same species but are incorporated into recipient cells by genetic engineering methods rather than classical reproduction or breeding techniques. However, the term “exogenous” is also intended to refer to genes that are not normally present in the cell being transformed, or perhaps simply not present in the form, structure, etc., as found in the transforming DNA segment or gene, or genes which are normally present and that one desires to express in a mamier that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the plant cell, DNA from another plant, DNA from a different organ-ism, or a DNA generated externally, such as a DNA sequence containing an antisense message of a gene, or a DNA sequence encoding a synthetic or modified version of a gene.

Many hundreds if not thousands of different genes are known and could potentially be introduced into a lettuce plant according to the invention. Non-limiting examples of particular genes and corresponding phenotypes one may choose to introduce into a lettuce plant include one or more genes for insect tolerance, such as a Bacillus thuringiensis (B.t.) gene, pest tolerance such as genes for fungal disease control, herbicide tolerance such as genes conferring gly-phosate tolerance, and genes for quality improvements such as yield, nutritional enhancements, environmental or stress tolerances, or any desirable changes in plant physiology, growth, development, morphology or plant product(s). For example, structural genes would include any gene that confers insect tolerance including but not limited to a Bacillus insect control protein gene as described in WO 99/31248, herein incorporated by reference in its entirety, U.S. Pat. No. 5,689,052, herein incorporated by reference in its entirety, U.S. Pat. Nos. 5,500,365 and 5,880,275, herein incorporated by reference in their entirety. In another embodiment, the structural gene can confer tolerance to the herbicide glyphosate as conferred by genes including, but not limited to Agrobacterium strain CP4 glyphosate resistant EPSPS gene (aroA:CP4) as described in U.S. Pat. No. 5,633,435, herein incorporated by reference in its entirety, or glyphosate oxidoreductase gene (GOX) as described in U.S. Pat. No. 5,463,175, herein incorporated by reference in its entirety.

Alternatively, the DNA coding sequences can affect these phenotypes by encoding a non-translatable RNA molecule that causes the targeted inhibition of expression of an endogenous gene, for example via antisense- or cosuppression-mediated mechanisms (see, for example, Bird et al., Biotech. Gen. Engin. Rev., 9:207, 1991). The RNA could also be a catalytic RNA molecule (i.e., a ribozyme) engineered to cleave a desired endogenous mRNA product (see, for example, Gibson and Shillito, Mal. Biotech., 7: 125, 1997). Thus, any gene which produces a protein or mRNA which expresses a phenotype or morphology change of interest is useful for the practice of the present invention.

### INCORPORATION BY REFERENCE

The nucleotide and amino acid sequences disclosed herein are provided in a separate Sequence Listing submitted concurrently with this application and are hereby incorporated by reference in their entirety. This Sequence Listing is formatted in accordance with the requirements of 37 CFR 1.831-1.835 and is available as an electronic file, identified as 1715-010 Lettuce Fusarium Resistance.xml, which can be accessed through the USPTO electronic filing system. The sequence listing was created on Sep. 11, 2024. The size of the XML file is 36,907 bytes.

### Further Embodiments of the Invention

With the advent of molecular biological techniques that have allowed the isolation and characterization of genes that encode specific protein products, scientists in the field of plant biology developed a strong interest in engineering the genome of plants (genetic engineering) to contain and express foreign genes, or additional, or modified versions of native, or endogenous, genes (perhaps driven by different promoters) in order to alter the traits of a plant in a specific manner. Plants altered by genetic engineering are often referred to as ‘genetically modified’. Any sequences, such as DNA, whether from a different species or from the same species, which are stably inserted into the cell using transformation are referred to herein collectively as “transgenes” and/or “transgenic events”. Transgenes can be moved from one genome to another using breeding techniques which may include crossing, backcrossing or double haploid production. In some embodiments of the invention, a transgenic variant of lettuce plants that produce seed having reduced-fat content may contain at least one transgene but could contain at least 1, 2, 3, 4, 5, 6, 7, 8, 9, 10 and/or no more than 15, 14, 13, 12, 11, 10, 9, 8, 7, 6, 5, 4, 3, or 2. Over the last fifteen to twenty years, several methods for producing transgenic plants have been developed, and the present invention, in particular embodiments, also relates to transformed versions of the claimed cultivar.

Culture for expressing desired structural genes and cultured cells is known in the art. Also as known in the art, lettuce is transformable and regenerable such that whole plants containing and expressing desired genes under regulatory control may be obtained. General descriptions of plant expression vectors and reporter genes and transformation protocols can be found in Gruber, et al., “Vectors for Plant Transformation”, in Methods in Plant Molecular Biology & Biotechnology in Glich, et al., (Eds. pp. 89-119, CRC Press, 1993). Moreover, GUS expression vectors and GUS gene cassettes are available from Clone Tech Laboratories, Inc., Palo Alto, California while luciferase expression vectors and luciferase gene cassettes are available from Pro Mega Corp. (Madison, Wisconsin). Methods for transformation and regeneration of lettuce cells are known in the art.

General methods of culturing plant tissues are provided for example by Maki, et al., “Procedures for Introducing Foreign DNA into Plants” in Methods in Plant Molecular Biology & Biotechnology, Glich, et al., (Eds. pp. 67-88 CRC Press, 1993); and by Phillips, et al., “Cell-Tissue Culture and In-Vitro Manipulation” in Corn & Corn Improvement, 3rd Edition; Sprague, et al., (Eds. pp. 345-387 American Society of Agronomy Inc., 1988). Methods of introducing expression vectors into plant tissue include the direct infection or co-cultivation of plant cells with Agrobacterium tumefaciens, described for example by Horsch et al., Science, 227:1229 (1985). Descriptions of Agrobacterium vector systems and methods for Agrobacterium-mediated gene transfer are provided by Gruber, et al., supra.

Useful methods include but are not limited to expression vectors introduced into plant tissues using a direct gene transfer method such as microprojectile-mediated delivery, DNA injection, electroporation and the like. More preferably expression vectors are introduced into plant tissues using a microprojectile media delivery system with a biolistic device or using Agrobacterium-mediated transformation. Transformant plants obtained with the protoplasm of the invention are intended to be within the scope of this invention.

Plant transformation involves the construction of an expression vector which will function in plant cells. Such a vector comprises DNA comprising a gene under control of or operatively linked to a regulatory element (for example, a promoter). The expression vector may contain one or more such operably linked gene/regulatory element combinations. The vector(s) may be in the form of a plasmid and can be used alone or in combination with other plasmids, to provide transformed lettuce plants, using transformation methods as described below to incorporate transgenes into the genetic material of the lettuce plant(s).

One embodiment of the invention is a process for producing a lettuce line of the present invention further comprising a desired trait, said process comprising introducing a transgene that confers a desired trait to a lettuce plant having resistance to Fusarium wilt of the present invention. In one embodiment the desired trait may be one or more of herbicide resistance, insect resistance, disease resistance, decreased phytate, or modified fatty acid or carbohydrate metabolism. The specific gene may be any known in the art or listed herein, including: a polynucleotide conferring resistance to imidazolinone, dicamba, sulfonylurea, glyphosate, glufosinate, triazine, benzonitrile, cyclohexanedione, phenoxy proprionic acid, and L-phosphinothricin; a polynucleotide encoding a Bacillus thuringiensis polypeptide; a polynucleotide encoding phytase, FAD-2, FAD-3, galactinol synthase, or a raffinose synthetic enzyme; or a polynucleotide conferring resistance to rust (Puccinia arachidis), early and late leaf spot (Cercospora arachidicola and Cercosporidium personatum), web blotch (Didymella arachidicola), pepper spot (Leptosphaerulina crassiasca), TSWV, atmospheric scorch, chemical burn, iron chlorosis, potato leafhopper (Empoasca fabae) seedling disease (Rhizoctonia solani, Pythium spp., Fusarium spp. and others), yellow mold (Aspergillus flavus, Aspergillus parasiticus), root knot nematode (Meloidogyne arenaria) root lesion nematode, southern blight (Sclerotium rolfsii), sclerotinia blight (Sclerotinia minor), Rhizoctonia pod, peg and limb rot (Rhizoctonia solani), pythium pod rot (Pythium myriotylum), botrytis blight (Botrytis cinerea), black mold (Aspergillus niger), blackhull (Thielaviopsis basicola), Phymatotrichum root rot (Phymatotrichum omnivorum), and tooth fungus (Phanerochaeta sp.).

### Expression Vectors for Transformation: Marker Genes

Expression vectors include at least one genetic marker, operably linked to a regulatory element (a promoter, for example) that allows transformed cells containing the marker to be either recovered by negative selection, i.e., inhibiting growth of cells that do not contain the selectable marker gene, or by positive selection, i.e., screening for the product encoded by the genetic marker. Many commonly used selectable marker genes for plant transformation are well known in the transformation arts, and include, for example, genes that code for enzymes that metabolically detoxify a selective chemical agent which may be an antibiotic or an herbicide, or genes that encode an altered target which is insensitive to the inhibitor. A few positive selection methods are also known in the art.

One commonly used selectable marker gene for plant transformation is the neomycin phosphotransferase II (nptII) gene, isolated from transposon Tn5, which when placed under the control of plant regulatory signals confers resistance to kanamycin. Fraley et al., Proc. Natl. Acad. Sci. U.S.A., 80:4803 (1983). Another commonly used selectable marker gene is the hygromycin phosphotransferase gene which confers resistance to the antibiotic hygromycin. Vanden Elzen et al., Plant Mol. Biol., 5:299 (1985).

Additional selectable marker genes of bacterial origin that confer resistance to antibiotics include gentamycin acetyl transferase, streptomycin phosphotransferase, and aminoglycoside-3′-adenyl transferase, the bleomycin resistance determinant. Hayford et al., Plant Physiol. 86:1216 (1988); Jones et al., Mol. Gen. Genet., 210:86 (1987); Svab et al., Plant Mol. Biol. 14:197 (1990); Hille et al., Plant Mol. Biol. 7:171 (1986). Other selectable marker genes confer resistance to herbicides such as glyphosate, glufosinate or bromoxynil. Comai et al., Nature 317:741-744 (1985), Gordon-Kamm et al., Plant Cell 2:603-618 (1990) and Stalker et al., Science 242:419-423 (1988).

Selectable marker genes for plant transformation not of bacterial origin include, for example, mouse dihydrofolate reductase, plant 5-enolpyruvylshikimate-3-phosphate synthase and plant acetolactate synthase. Eichholtz et al., Somatic Cell Mol. Genet. 13:67 (1987), Shah et al., Science 233:478 (1986), Charest et al., Plant Cell Rep. 8:643 (1990).

Another class of marker genes for plant transformation requires screening of presumptively transformed plant cells rather than direct genetic selection of transformed cells for resistance to a toxic substance such as an antibiotic. These genes are particularly useful to quantify or visualize the spatial pattern of expression of a gene in specific tissues and are frequently referred to as reporter genes because they can be fused to a gene or gene regulatory sequence for the investigation of gene expression. Commonly used genes for screening presumptively transformed cells include β-glucuronidase (GUS, β-galactosidase, luciferase and chloramphenicol acetyltransferase. Jefferson, R. A., Plant Mol. Biol. Rep. 5:387 (1987), Teeri et al., EMBO J. 8:343 (1989), Koncz et al., Proc. Natl. Acad. Sci U.S.A. 84:131 (1987), DeBlock et al., EMBO J. 3:1681 (1984). Another approach to the identification of relatively rare transformation events has been use of a gene that encodes a dominant constitutive regulator of the Zea mays anthocyanin pigmentation pathway Ludwig et al., Science 247:449 (1990).

In vivo methods for visualizing GUS activity that do not require destruction of plant tissue are available. Molecular Probes publication 2908, IMAGENE GREEN, p. 1-4 (1993) and Naleway et al., J. Cell Biol. 115:151a (1991). However, these in vivo methods for visualizing GUS activity have not proven useful for recovery of transformed cells because of low sensitivity, high fluorescent backgrounds and limitations associated with the use of luciferase genes as selectable markers.

More recently, a gene encoding Green Fluorescent Protein (GFP) has been utilized as a marker for gene expression in prokaryotic and eukaryotic cells. Chalfie et al., Science 263:802 (1994). GFP and mutants of GFP may be used as screenable markers.

### Expression Vectors for Transformation: Promoters

Genes included in expression vectors must be driven by nucleotide sequence comprising a regulatory element, for example, a promoter. Several types of promoters are now well known in the transformation arts, as are other regulatory elements that can be used alone or in combination with promoters.

As used herein, “promoter” includes reference to a region of DNA upstream from the start of transcription and involved in recognition and binding of RNA polymerase and other proteins to initiate transcription. A “plant promoter” is a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred”. Promoters which initiate transcription only in certain tissues are referred to as “tissue-specific”. A “cell type” specific promoter primarily drives expression in certain cell types in one or more organs, for example, vascular cells in roots or leaves. An “inducible” promoter is a promoter which is under environmental control. Examples of environmental conditions that may affect transcription by inducible promoters include anaerobic conditions or the presence of light. Tissue-specific, tissue-preferred, cell type specific, and inducible promoters constitute the class of “non-constitutive” promoters. A “constitutive” promoter is a promoter which is active under most environmental conditions.

A. Inducible Promoters. An inducible promoter is operably linked to a gene for expression in lettuce. Optionally, the inducible promoter is operably linked to a nucleotide sequence encoding a signal sequence which is operably linked to a gene for expression in lettuce. With an inducible promoter the rate of transcription increases in response to an inducing agent.

Any inducible promoter can be used in the instant invention. See Ward et al., Plant Mol. Biol. 22:361-366 (1993). Exemplary inducible promoters include, but are not limited to, that from the ACEI system which responds to copper (Meft et al., PNAS 90:4567-4571 (1993)); In2 gene from maize which responds to benzenesulfonamide herbicide safeners (Hershey et al., Mol. Gen Genetics 227:229-237 (1991) and Gatz et al., Mol. Gen. Genetics 243:32-38 (1994)) or Tet repressor from Tn10 (Gatz et al., Mol. Gen. Genetics 227:229-237 (1991)). A particularly preferred inducible promoter is a promoter that responds to an inducing agent to which plants do not normally respond. An exemplary inducible promoter is the inducible promoter from a steroid hormone gene, the transcriptional activity of which is induced by a glucocorticosteroid hormone (Schena et al., Proc. Natl. Acad. Sci. U.S.A. 88:0421 (1991)).

B. Constitutive Promoters—A constitutive promoter is operably linked to a gene for expression in lettuce or the constitutive promoter is operably linked to a nucleotide sequence encoding a signal sequence which is operably linked to a gene for expression in lettuce.

Many different constitutive promoters can be utilized in the instant invention. Exemplary constitutive promoters include, but are not limited to, the promoters from plant viruses such as the 35S promoter from CaMV (Odell et al., Nature 313:810-812 (1985)) and the promoters from such genes as rice actin (McElroy et al., Plant Cell 2:163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)) and maize H3 histone (Lepetit et al., Mol. Gen. Genetics 231:276-285 (1992) and Atanassova et al., Plant Journal 2 (3): 291-300 (1992)).

The ALS promoter, Xba1/Nco1 fragment 5′ to the Brassica napus ALS3 structural gene (or a nucleotide sequence similarity to said Xba1/Nco1 fragment), represents a particularly useful constitutive promoter. See PCT application WO96/30530.

C. Tissue-specific or Tissue-preferred Promoters—A tissue-specific promoter is operably linked to a gene for expression in lettuce. Optionally, the tissue-specific promoter is operably linked to a nucleotide sequence encoding a signal sequence which is operably linked to a gene for expression in lettuce. Plants transformed with a gene of interest operably linked to a tissue-specific promoter produce the protein product of the transgene exclusively, or preferentially, in a specific tissue.

Any tissue-specific or tissue-preferred promoter can be utilized in the instant invention. Exemplary tissue-specific or tissue-preferred promoters include, but are not limited to, a root-preferred promoter, such as that from the phaseolin gene (Murai et al., Science 23:476-482 (1983) and Sengupta-Gopalan et al., Proc. Natl. Acad. Sci. U.S.A. 82:3320-3324 (1985)); a leaf-specific and light-induced promoter such as that from cab or rubisco (Simpson et al., EMBO J. 4(11):2723-2729 (1985) and Timko et al., Nature 318:579-582 (1985)); an anther-specific promoter such as that from LAT52 (Twell et al., Mol. Gen. Genetics 217:240-245 (1989)); a pollen-specific promoter such as that from Zm13 (Guerrero et al., Mol. Gen. Genetics 244:161-168 (1993)) or a microspore-preferred promoter such as that from apg (Twell et al., Sex. Plant Reprod. 6:217-224 (1993)).

### Signal Sequences for Targeting

### Proteins to Subcellular Compartments

Transport of protein produced by transgenes to a subcellular compartment such as the chloroplast, vacuole, peroxisome, glyoxysome, cell wall or mitochondrion or for secretion into the apoplast, is accomplished by means of operably linking the nucleotide sequence encoding a signal sequence to the 5′ and/or 3′ region of a gene encoding the protein of interest. Targeting sequences at the 5′ and/or 3′ end of the structural gene may determine, during protein synthesis and processing, where the encoded protein is ultimately compartmentalized.

The presence of a signal sequence directs a polypeptide to either an intracellular organelle or subcellular compartment or for secretion to the apoplast. Many signal sequences are known in the art. See, for example Becker et al., Plant Mol. Biol. 20:49 (1992); Close, P. S., Master's Thesis, Iowa State University (1993); Knox, C., et al., Plant Mol. Biol. 9:3-17 (1987); Lerner et al., Plant Physiol. 91:124-129 (1989); Fontes et al., Plant Cell 3:483-496 (1991); Matsuoka et al., Proc. Natl. Acad. Sci. 88:834 (1991); Gould et al., J. Cell. Biol. 108:1657 (1989); Creissen et al., Plant J. 2:129 (1991); Kalderon, et al., Cell 39:499-509 (1984); Steifel, et al., Plant Cell 2:785-793 (1990).

### Foreign Protein Genes and Agronomic Genes

With transgenic plants according to the present invention, a foreign protein can be produced in commercial quantities. Thus, techniques for the selection and propagation of transformed plants, which are well understood in the art, yield a plurality of transgenic plants which are harvested in a conventional manner, and a foreign protein then can be extracted from a tissue of interest or from total biomass. Protein extraction from plant biomass can be accomplished by known methods which are discussed, for example, by Heney and Orr, Anal. Biochem. 114:92-6 (1981).

According to an embodiment, the transgenic plant provided for commercial production of foreign protein is lettuce. In another embodiment, the biomass of interest is seed. For the relatively small number of transgenic plants that show higher levels of expression, a genetic map can be generated, primarily via conventional RFLP, PCR and SSR analysis, which identifies the approximate chromosomal location of the integrated DNA molecule. For exemplary methodologies in this regard, see Glick and Thompson, Methods in Plant Molecular Biology and Biotechnology CRC Press, Boca Raton 269:284 (1993).

Map information concerning chromosomal location is useful for proprietary protection of a subject transgenic plant. If unauthorized propagation is undertaken and crosses made with other germplasm, the map of the integration region can be compared to similar maps for suspect plants, to determine if the latter have a common parentage with the subject plant. Map comparisons would involve hybridizations, RFLP, PCR, SSR and sequencing, all of which are conventional techniques.

Through the transformation of lettuce, the expression of genes can be altered to enhance disease resistance, insect resistance, herbicide resistance, agronomic quality and other traits. Transformation can also be used to insert DNA sequences which control or help control male-sterility. DNA sequences native to lettuce as well as non-native DNA sequences can be transformed into lettuce and used to alter levels of native or non-native proteins. Various promoters, targeting sequences, enhancing sequences, and other DNA sequences can be inserted into the genome for the purpose of altering the expression of proteins. Reduction of the activity of specific genes (also known as gene silencing, or gene suppression) is desirable for several aspects of genetic engineering in plants.

Many techniques for gene silencing are well known to one of skill in the art, including but not limited to knock-outs (such as by insertion of a transposable element such as mu (Vicki Chandler, The Maize Handbook ch. 118 (Springer-Verlag 1994) or other genetic elements such as a FRT, Lox or other site specific integration site, antisense technology (see, e.g., Sheehy et al. (1988) PNAS USA 85:8805-8809; and U.S. Pat. Nos. 5,107,065; 5,453,566; and 5,759,829); cosuppression (e.g., Taylor (1997) Plant Cell 9:1245; Jorgensen (1990) Trends Biotech. 8(12):340-344; Flavell (1994) PNAS USA 91:3490-3496; Finnegan et al. (1994) Bio/Technology 12: 883-888; and Neuhuber et al. (1994) Mol. Gen. Genet. 244:230-241); RNA interference (Napoli et al. (1990) Plant Cell 2:279-289; U.S. Pat. No. 5,034,323; Sharp (1999) Genes Dev. 13:139-141; Zamore et al. (2000) Cell 101:25-33; and Montgomery et al. (1998) PNAS USA 95:15502-15507), virus-induced gene silencing (Burton, et al. (2000) Plant Cell 12:691-705; and Baulcombe (1999) Curr. Op. Plant Bio. 2:109-113); target-RNA-specific ribozymes (Haseloff et al. (1988) Nature 334: 585-591); hairpin structures (Smith et al. (2000) Nature 407:319-320; WO 99/53050; and WO 98/53083); MicroRNA (Aukerman& Sakai (2003) Plant Cell 15:2730-2741); ribozymes (Steinecke et al. (1992) EMBO J. 11:1525; and Perriman et al. (1993) Antisense Res. Dev. 3:253); oligonucleotide mediated targeted modification (e.g., WO 03/076574 and WO 99/25853); Zn-finger targeted molecules (e.g., WO 01/52620; WO 03/048345; and WO 00/42219); and other methods or combinations of the above methods known to those of skill in the art.

Likewise, by means of the present invention, agronomic genes can be expressed in transformed plants. More particularly, plants can be genetically engineered to express various phenotypes of agronomic interest. Exemplary genes implicated in this regard include, but are not limited to, those categorized below:

1. Genes that Confer Resistance to Pests or Disease and that Encode:

A. Plant disease resistance genes. Plant defenses are often activated by specific interaction between the product of a disease resistance gene (R) in the plant and the product of a corresponding avirulence (Avr) gene in the pathogen. A plant cultivar can be transformed with cloned resistance gene to engineer plants that are resistant to specific pathogen strains. See, for example Jones et al., Science 266:789 (1994) (cloning of the lettuce Cf-9 gene for resistance to Cladosporium fulvum); Martin et al., Science 262:1432 (1993) (lettuce Pto gene for resistance to Pseudomonas syringae pv. lettuce encodes a protein kinase); Mindrinos et al., Cell 78:1089 (1994) (Arabidopsis RSP2 gene for resistance to Pseudomonas syringae).

B. A Bacillus thuringiensis protein, a derivative thereof or a synthetic polypeptide modeled thereon. See, for example, Geiser et al., Gene 48:109 (1986), who disclose the cloning and nucleotide sequence of a Bt δ-endotoxin gene. Moreover, DNA molecules encoding 6-endotoxin genes can be purchased from American Type Culture Collection, Manassas, Virginia, for example, under ATCC Accession Nos. 40098, 67136, 31995 and 31998.

C. A lectin. See, for example, the disclosure by Van Damme et al., Plant Molec. Biol. 24:25 (1994), who disclose the nucleotide sequences of several Clivia miniata mannose-binding lectin genes.

D. A vitamin-binding protein such as avidin. See PCT application US93/06487. The application teaches the use of avidin and avidin homologues as larvicides against insect pests.

E. An enzyme inhibitor, for example, a protease or proteinase inhibitor or an amylase inhibitor. See, for example, Abe et al., J. Biol. Chem. 262:16793 (1987) (nucleotide sequence of rice cysteine proteinase inhibitor), Huub et al., Plant Molec. Biol. 21:985 (1993) (nucleotide sequence of cDNA encoding tobacco proteinase inhibitor I), Sumitani et al., Biosci. Biotech. Biochem. 57:1243 (1993) (nucleotide sequence of Streptomyces nitrosporeus α-amylase inhibitor).

F. An insect-specific hormone or pheromone such as an ecdysteroid and juvenile hormone, a variant thereof, a mimetic based thereon, or an antagonist or agonist thereof. See, for example, the disclosure by Hammock et al., Nature 344:458 (1990), of baculovirus expression of cloned juvenile hormone esterase, an inactivator of juvenile hormone.

G. An insect-specific peptide or neuropeptide which, upon expression, disrupts the physiology of the affected pest. For example, see the disclosures of Regan, J. Biol. Chem. 269:9 (1994) (expression cloning yields DNA coding for insect diuretic hormone receptor), and Pratt et al., Biochem. Biophys. Res. Comm. 163:1243 (1989) (an allostatin is identified in Diploptera puntata); Chattopadhyay, et al., Critical Reviews in Microbiology, 30(1):33-54 (2004); Zjawiony, J Nat Prod, 67(2):300-310 (2004); Carlini & Grossi-de-Sa, Toxicon, 40(11):1515-1539 (2002); Ussuf, et al., Curr Sci., 80(7):847-853 (2001); Vasconcelos & Oliveira, Toxicon, 44(4):385-403 (2004). See also U.S. Pat. No. 5,266,317 to Tomalski et al., who disclose genes encoding insect-specific, paralytic neurotoxins.

H. An insect-specific venom produced in nature by a snake, a wasp, etc. For example, see Pang et al., Gene 116:165 (1992), for disclosure of heterologous expression in plants of a gene coding for a scorpion insectotoxic peptide.

I. An enzyme responsible for a hyper-accumulation of a monoterpene, a sesquiterpene, a steroid, a hydroxamic acid, a phenylpropanoid derivative or another non-protein molecule with insecticidal activity.

J. An enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. See PCT application WO 93/02197 in the name of Scott et al., which discloses the nucleotide sequence of a callase gene. DNA molecules which contain chitinase-encoding sequences can be obtained, for example, from the ATCC under Accession Nos. 39637 and 67152. See also Kramer et al., Insect Biochem. Molec. Biol. 23:691 (1993), who teach the nucleotide sequence of a cDNA encoding tobacco hornworm chitinase, and Kawalleck et al., Plant Molec. Biol. 21:673 (1993), who provide the nucleotide sequence of the parsley ubi4-2 polyubiquitin gene, U.S. Pat. Nos. 7,145,060, 7,087,810, and 6,563,020.

K. A molecule that stimulates signal transduction. For example, see the disclosure by Botella et al., Plant Molec. Biol. 24:757 (1994), of nucleotide sequences for mung bean calmodulin cDNA clones, and Griess et al., Plant Physiol. 104:1467 (1994), who provide the nucleotide sequence of a maize calmodulin cDNA clone.

L. A hydrophobic moment peptide. See PCT application WO95/16776 (disclosure of peptide derivatives of Tachyplesin which inhibit fungal plant pathogens) and PCT application WO95/18855 (teaches synthetic antimicrobial peptides that confer disease resistance), the respective contents of which are hereby incorporated by reference.

M. A membrane permease, a channel former or a channel blocker. For example, see the disclosure of Jaynes et al., Plant Sci 89:43 (1993), of heterologous expression of a cecropin-β, lytic peptide analog to render transgenic tobacco plants resistant to Pseudomonas solanacearum.

N. A viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. See Beachy et al., Ann. Rev. Phytopathol. 28:451 (1990). Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus. Id.

O. An insect-specific antibody or an immunotoxin derived therefrom. Thus, an antibody targeted to a critical metabolic function in the insect gut would inactivate an affected enzyme, killing the insect. Cf. Taylor et al., Abstract #497, Seventh Int'l Symposium on Molecular Plant-Microbe Interactions (Edinburgh, Scotland) (1994) (enzymatic inactivation in transgenic tobacco via production of single-chain antibody fragments.

P. A virus-specific antibody. See, for example, Tavladoraki et al., Nature 366:469 (1993), who show that transgenic plants expressing recombinant antibody genes are protected from virus attack.

Q. A developmental-arrestive protein produced in nature by a pathogen or a parasite. Thus, fungal endo-α-1, 4-D-polygalacturonases facilitate fungal colonization and plant nutrient release by solubilizing plant cell wall homo-α-1,4-D-galacturonase. See Lamb et al., Bio/Technology 10:1436 (1992). The cloning and characterization of a gene which encodes a bean endopolygalacturonase-inhibiting protein is described by Toubart et al., Plant J. 2:367 (1992).

R. A developmental-arrestive protein produced in nature by a plant. For example, Logemann et al., Bio/Technology 10:305 (1992), have shown that transgenic plants expressing the barley ribosome-inactivating gene have an increased resistance to fungal disease.

S. Genes involved in the Systemic Acquired Resistance (SAR) Response and/or the pathogenesis-related genes. Briggs, S., Current Biology, 5(2) (1995); Pieterse & Van Loon, Curr. Opin. Plant Bio., 7(4):456-64 (2004); and Somssich, Cell, 113(7):815-6 (2003).

T. Antifungal genes. See, Cornelissen and Melchers, Plant Physiol., 101:709-712 (1993); Parijs, et al., Planta, 183:258-264 (1991); and Bushnell, et al., Can. J. of Plant Path., 20(2):137-149 (1998). See also, U.S. Pat. No. 6,875,907.

U. Detoxification genes, such as for fumonisin, beauvericin, moniliformin, and zearalenone and their structurally-related derivatives. See, for example, U.S. Pat. No. 5,792,931.

V. Cystatin and cysteine proteinase inhibitors. See, U.S. Pat. No. 7,205,453.

W. Defensin genes. See, WO 03/000863 and U.S. Pat. No. 6,911,577.

X. Genes conferring resistance to nematodes, such as root knot nematode and root lesion nematode. See, e.g., PCT Applications WO 96/30517, WO 93/19181, and WO 03/033651; Urwin, et al., Planta, 204:472-479 (1998); Williamson, Curr Opin Plant Bio., 2(4):327-31 (1999).

Y. Genes that confer resistance to Phytophthora Root Rot, such as the Rps 1, Rps 1-a, Rps 1-b, Rps 1-c, Rps 1-d, Rps 1-e, Rps 1-k, Rps 2, Rps 3-a, Rps 3-b, Rps 3-c, Rps 4, Rps 5, Rps 6, Rps 7, and other Rps genes. See, for example, Shoemaker, et al., Phytophthora Root Rot Resistance Gene Mapping in Soybean, Plant Genome IV Conference, San Diego, Calif (1995).

Z. Genes that confer resistance to Brown Stem Rot, such as described in U.S. Pat. No. 5,689,035 and incorporated by reference for this purpose.

Any of the above-listed disease or pest resistance genes (A-Z) can be introduced into a claimed lettuce line of the invention through a variety of means including, but not limited to, transformation and crossing.

2. Genes that Confer Resistance to an Herbicide, for Example:

A. An herbicide that inhibits the growing point or meristem, such as an imidazolinone, or a sulfonylurea. Exemplary genes in this category code for mutant ALS and AHAS enzymes as described, for example, by Lee et al., EMBO J. 7:1241 (1988), and Miki et al., Theor. Appl. Genet. 80:449 (1990), respectively. Additionally, a meristematic inhibitor herbicide such as pendimethalin.

B. Glyphosate (resistance conferred by mutant 5-enolpyruvlshikimate-3-phosphate synthase (EPSP) and aroA genes, respectively) and other phosphono compounds such as glufosinate (phosphinothricin acetyl transferase (PAT) and Streptomyces hygroscopicus PAT, bar, genes), and pyridinoxy or phenoxy propionic acids and cyclohexones, as well as herbicides that inhibit the enzyme acetyl-CoA carboxylase (ACCase inhibitor-encoding genes). See, for example, U.S. Pat. No. 4,940,835 to Shah, et al., which discloses the nucleotide sequence of a form of EPSP which can confer glyphosate resistance. A DNA molecule encoding a mutant aroA gene can be obtained under ATCC accession number 39256, and the nucleotide sequence of the mutant gene is disclosed in U.S. Pat. No. 4,769,061 to Comai. European patent application No. 0 333 033 to Kumada et al., and U.S. Pat. No. 4,975,374 to Goodman et al., disclose nucleotide sequences of glutamine synthetase genes which confer resistance to herbicides such as L-phosphinothricin. The nucleotide sequence of a PAT gene is provided in European application No. 0 242 246 to Leemans et al. DeGreef et al., Bio/Technology 7:61 (1989), describe the production of transgenic plants that express chimeric bar genes coding for PAT activity. Exemplary of genes conferring resistance to phenoxy propionic acids and cyclohexones, such as sethoxydim and haloxyfop are the Acc1-S1, Acc1-S2 and Acc1-S3 genes described by Marshall et al., Theor. Appl. Genet. 83:435 (1992).

C. An herbicide that inhibits photosynthesis, such as a triazine (psbA and gs+ genes) or a benzonitrile (nitrilase gene). Przibilla et al., Plant Cell 3:169 (1991), describe the transformation of Chlamydomonas with plasmids encoding mutant psbA genes. Nucleotide sequences for nitrilase genes are disclosed in U.S. Pat. No. 4,810,648 to Stalker, and DNA molecules containing these genes are available under ATCC Accession Nos. 53435, 67441, and 67442. Cloning and expression of DNA coding for a glutathione S-transferase is described by Hayes et al., Biochem. J. 285:173 (1992)

D. Acetohydroxy acid synthase, which has been found to make plants that express this enzyme resistant to multiple types of herbicides, has been introduced into a variety of plants. See, Hattori, et al., Mol. Gen. Genet., 246:419 (1995). Other genes that confer tolerance to herbicides include a gene encoding a chimeric protein of rat cytochrome P4507A1 and yeast NADPH-cytochrome P450 oxidoreductase (Shiota, et al., Plant Physiol., 106:17 (1994)); genes for glutathione reductase and superoxide dismutase (Aono, et al., Plant Cell Physiol., 36:1687 (1995)); and genes for various phosphotransferases (Datta, et al., Plant Mol. Biol., 20:619 (1992)).

E. Protoporphyrinogen oxidase (protox) is necessary for the production of chlorophyll, which is necessary for all plant survival. The protox enzyme serves as the target for a variety of herbicidal compounds. These herbicides also inhibit growth of all the different species of plants present, causing their total destruction. The development of plants containing altered protox activity which are resistant to these herbicides are described in U.S. Pat. Nos. 6,288,306, 6,282,837, 5,767,373, and International Publication WO 01/12825.

Any of the above listed herbicide genes (A-E) can be introduced into a claimed reduced-fat lettuce line of the present invention through a variety of means including but not limited to transformation and crossing.

3. Genes that Confer or Contribute to a Value-Added Trait, Such as:

A. Modified fatty acid metabolism, for example, by transforming a plant with an antisense gene of stearyl-ACP desaturase to increase stearic acid content of the plant. See Knultzon et al., Proc. Natl. Acad. Sci. U.S.A. 89:2624 (1992).

B. Decreased phytate content, 1) Introduction of a phytase-encoding gene would enhance breakdown of phytate, adding more free phosphate to the transformed plant. For example, see Van Hartingsveldt et al., Gene 127:87 (1993), for a disclosure of the nucleotide sequence of an Aspergillus niger phytase gene; 2) A gene could be introduced that reduced phytate content. In maize, this, for example, could be accomplished, by cloning and then reintroducing DNA associated with the single allele which is responsible for maize mutants characterized by low levels of phytic acid. See Raboy et al., Maydica 35:383 (1990).

C. Modified carbohydrate composition effected, for example, by transforming plants with a gene coding for an enzyme that alters the branching pattern of starch. See Shiroza et al., J. Bacteol. 170:810 (1988) (nucleotide sequence of Streptococcus mutants fructosyltransferase gene), Steinmetz et al., Mol. Gen. Genet. 20:220 (1985) (nucleotide sequence of Bacillus subtilis levansucrase gene), Pen et al., Bio/Technology 10:292 (1992) (production of transgenic plants that express Bacillus lichenifonnis α-amylase), Elliot et al., Plant Molec. Biol. 21:515 (1993) (nucleotide sequences of lettuce invertase genes), Søgaard et al., J. Biol. Chem. 268:22480 (1993) (site-directed mutagenesis of barley α-amylase gene), and Fisher et al., Plant Physiol. 102:1045 (1993) (maize endosperm starch branching enzyme II).

D. Elevated oleic acid via FAD-2 gene modification and/or decreased linolenic acid via FAD-3 gene modification. See, U.S. Pat. Nos. 6,063,947, 6,323,392, and International Publication WO 93/11245.

E. Altering conjugated linolenic or linoleic acid content, such as in WO 01/12800. Altering LEC1, AGP, Dek1, Superall, mi1ps, and various Ipa genes, such as Ipa1, Ipa3, hpt, or hggt. See, for example, WO 02/42424, WO 98/22604, WO 03/011015, WO 02/057439, WO 03/011015, U.S. Pat. Nos. 6,423,886, 6,197,561, 6,825,397, 7,157,621, U.S. Publ. No. 2003/0079247, and Rivera-Madrid, R., et al., Proc. Natl. Acad. Sci., 92:5620-5624 (1995).

F. Altered antioxidant content or composition, such as alteration of tocopherol or tocotrienols. See, for example, U.S. Pat. Nos. 6,787,683, 7,154,029, WO 00/68393 (involving the manipulation of antioxidant levels through alteration of a phytl prenyl transferase (ppt)); WO 03/082899 (through alteration of a homogentisate geranyl geranyl transferase (hggt)).

G. Altered essential seed amino acids. See, for example, U.S. Pat. No. 6,127,600 (method of increasing accumulation of essential amino acids in seeds); U.S. Pat. No. 6,080,913 (binary methods of increasing accumulation of essential amino acids in seeds); U.S. Pat. No. 5,990,389 (high lysine); U.S. Pat. No. 5,850,016 (alteration of amino acid compositions in seeds); U.S. Pat. No. 5,885,802 (high methionine); U.S. Pat. No. 5,885,801 (high threonine); U.S. Pat. No. 6,664,445 (plant amino acid biosynthetic enzymes); U.S. Pat. No. 6,459,019 (increased lysine and threonine); U.S. Pat. No. 6,441,274 (plant tryptophan synthase beta subunit); U.S. Pat. No. 6,346,403 (methionine metabolic enzymes); U.S. Pat. No. 5,939,599 (high sulfur); U.S. Pat. No. 5,912,414 (increased methionine); U.S. Pat. No. 5,633,436 (increasing sulfur amino acid content); U.S. Pat. No. 5,559,223 (synthetic storage proteins with defined structure containing programmable levels of essential amino acids for improvement of the nutritional value of plants); U.S. Pat. No. 6,194,638 (hemicellulose); U.S. Pat. No. 7,098,381 (UDPGdH); U.S. Pat. No. 6,194,638 (RGP); U.S. Pat. Nos. 6,399,859, 6,930,225, 7,179,955, and 6,803,498; U.S. Publ. No. 2004/0068767; WO 99/40209 (alteration of amino acid compositions in seeds); WO 99/29882 (methods for altering amino acid content of proteins); WO 98/20133 (proteins with enhanced levels of essential amino acids); WO 98/56935 (plant amino acid biosynthetic enzymes); WO 98/45458 (engineered seed protein having higher percentage of essential amino acids); WO 98/42831 (increased lysine); WO 96/01905 (increased threonine); WO 95/15392 (increased lysine); WO 01/79516; and WO 00/09706 (Ces A: cellulose synthase).

4. Genes that Control Male Sterility:

There are several methods of conferring genetic male sterility available, such as multiple mutant genes at separate locations within the genome that confer male sterility, as disclosed in U.S. Pat. Nos. 4,654,465 and 4,727,219 to Brar, et al., and chromosomal translocations as described by Patterson in U.S. Pat. Nos. 3,861,709 and 3,710,511. In addition to these methods, Albertsen, et al., U.S. Pat. No. 5,432,068, describes a system of nuclear male sterility which includes: identifying a gene which is critical to male fertility; silencing this native gene which is critical to male fertility; removing the native promoter from the essential male fertility gene and replacing it with an inducible promoter; inserting this genetically engineered gene back into the plant; and thus creating a plant that is male sterile because the inducible promoter is not “on” resulting in the male fertility gene not being transcribed. Fertility is restored by inducing, or turning “on,” the promoter, which in turn allows the gene that confers male fertility to be transcribed.

A. Introduction of a deacetylase gene under the control of a tapetum-specific promoter and with the application of the chemical N-Ac-PPT. See, International Publication WO 01/29237.

B. Introduction of various stamen-specific promoters. See, International Publications WO 92/13956 and WO 92/13957.

C. Introduction of the barnase and the barstar genes. See, Paul, et al., Plant Mol. Biol., 19:611-622 (1992).

For additional examples of nuclear male and female sterility systems and genes, see also, U.S. Pat. Nos. 5,859,341, 6,297,426, 5,478,369, 5,824,524, 5,850,014, and 6,265,640, all of which are hereby incorporated by reference.

5. Genes that Create a Site for Site Specific DNA Integration:

This includes the introduction of FRT sites that may be used in the FLP/FRT system and/or Lox sites that may be used in the Cre/Loxp system. See, for example, Lyznik, et al., Site-Specific Recombination for Genetic Engineering in Plants, Plant Cell Rep, 21:925-932 (2003) and WO 99/25821, which are hereby incorporated by reference. Other systems that may be used include the Gin recombinase of phage Mu (Maeser, et al. (1991); Vicki Chandler, The Maize Handbook, Ch. 118 (Springer-Verlag 1994)); the Pin recombinase of E. coli (Enomoto, et al. (1983)); and the R/RS system of the pSRl plasmid (Araki, et al. (1992)).

6. Genes that Affect Abiotic Stress Resistance:

Genes that affect abiotic stress resistance (including but not limited to flowering, pod and seed development, enhancement of nitrogen utilization efficiency, altered nitrogen responsiveness, drought resistance or tolerance, cold resistance or tolerance, and salt resistance or tolerance) and increased yield under stress. For example, see: WO 00/73475 where water use efficiency is altered through alteration of malate; U.S. Pat. Nos. 5,892,009, 5,965,705, 5,929,305, 5,891,859, 6,417,428, 6,664,446, 6,706,866, 6,717,034, 6,801,104, WO 2000/060089, WO 2001/026459, WO 2001/035725, WO 2001/034726, WO 2001/035727, WO 2001/036444, WO 2001/036597, WO 2001/036598, WO 2002/015675, WO 2002/017430, WO 2002/077185, WO 2002/079403, WO 2003/013227, WO 2003/013228, WO 2003/014327, WO 2004/031349, WO 2004/076638, WO 98/09521, and WO 99/38977 describing genes, including CBF genes and transcription factors effective in mitigating the negative effects of freezing, high salinity, and drought on plants, as well as conferring other positive effects on plant phenotype; U.S. Publ. No. 2004/0148654 and WO 01/36596, where abscisic acid is altered in plants resulting in improved plant phenotype, such as increased yield and/or increased tolerance to abiotic stress; WO 2000/006341, WO 04/090143, U.S. Pat. Nos. 7,531,723, and 6,992,237, where cytokinin expression is modified resulting in plants with increased stress tolerance, such as drought tolerance, and/or increased yield. See also, WO 02/02776, WO 2003/052063, JP 2002281975, U.S. Pat. No. 6,084,153, WO 01/64898, and U.S. Pat. Nos. 6,177,275 and 6,107,547 (enhancement of nitrogen utilization and altered nitrogen responsiveness). For ethylene alteration, see, U.S. Publ. Nos. 2004/0128719, 2003/0166197, and WO 2000/32761. For plant transcription factors or transcriptional regulators of abiotic stress, see, e.g., U.S. Publ. Nos. 2004/0098764 or 2004/0078852.

Other genes and transcription factors that affect plant growth and agronomic traits, such as yield, flowering, plant growth, and/or plant structure, can be introduced or introgressed into plants. See, e.g., WO 97/49811 (LHY), WO 98/56918 (ESD4), WO 97/10339, U.S. Pat. No. 6,573,430 (TFL), 6,713,663 (FT), 6,794,560, 6,307,126 (GAI), WO 96/14414 (CON), WO 96/38560, WO 01/21822 (VRN1), WO 00/44918 (VRN2), WO 99/49064 (GI), WO 00/46358 (FRI), WO 97/29123, WO 99/09174 (D8 and Rht), WO 2004/076638, and WO 004/031349 (transcription factors).

### Methods for Transformation

Numerous methods for plant transformation have been developed, including biological and physical, plant transformation protocols. See, for example, Miki et al., “Procedures for Introducing Foreign DNA into Plants” in Methods in Plant Molecular Biology and Biotechnology, Glick B. R. and Thompson, J. E. Eds. (CRC Press, Inc., Boca Raton, 1993) pages 67-88. In addition, expression vectors and in vitro culture methods for plant cell or tissue transformation and regeneration of plants are available. See, for example, Gruber et al., “Vectors for Plant Transformation” in Methods in Plant Molecular Biology and Biotechnology, Glick B. R. and Thompson, J. E. Eds. (CRC Press, Inc., Boca Raton, 1993) pages 89-119.

A. Agrobacterium-mediated Transformation—One method for introducing an expression vector into plants is based on the natural transformation system of Agrobacterium. See, for example, Horsch et al., Science 227:1229 (1985). A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. See, for example, Kado, C. I., Crit. Rev. Plant Sci. 10:1 (1991). Descriptions of Agrobacterium vector systems and methods for Agrobacterium-mediated gene transfer are provided by Gruber et al., supra, Miki et al., supra, and Moloney et al., Plant Cell Reports 8:238 (1989). See also, U.S. Pat. No. 5,591,616 issued Jan. 7, 1997.

B. Direct Gene Transfer—Despite the fact the host range for Agrobacterium-mediated transformation is broad, some major cereal crop species and gymnosperms have generally been recalcitrant to this mode of gene transfer, even though some success has recently been achieved in rice and corn. Hiei et al., The Plant Journal 6:271-282 (1994) and U.S. Pat. No. 5,591,616 issued Jan. 7, 1997. Several methods of plant transformation, collectively referred to as direct gene transfer, have been developed as an alternative to Agrobacterium-mediated transformation.

A generally applicable method of plant transformation is microprojectile-mediated transformation wherein DNA is carried on the surface of microprojectiles measuring 1 to 4 μm. The expression vector is introduced into plant tissues with a biolistic device that accelerates the microprojectiles to speeds of 300 to 600 m/s which is sufficient to penetrate plant cell walls and membranes. Sanford et al., Part. Sci. Technol. 5:27 (1987), Sanford, J. C., Trends Biotech. 6:299 (1988), Klein et al., Bio/Technology 6:559-563 (1988), Sanford, J. C., Physiol Plant 7:206 (1990), Klein et al., Biotechnology 10:268 (1992). In corn, several target tissues can be bombarded with DNA-coated microprojectiles in order to produce transgenic plants, including, for example, callus (Type I or Type II), immature embryos, and meristematic tissue.

Another method for physical delivery of DNA to plants is sonication of target cells. Zhang et al., Bio/Technology 9:996 (1991). Additionally, liposome and spheroplast fusion have been used to introduce expression vectors into plants. Deshayes et al., EMBO J., 4:2731 (1985), Christou et al., Proc Natl. Acad. Sci. U.S.A. 84:3962 (1987). Direct uptake of DNA into protoplasts using CaCl₂ precipitation, polyvinyl alcohol or poly-L-ornithine has also been reported. Hain et al., Mol. Gen. Genet. 199:161 (1985) and Draper et al., Plant Cell Physiol. 23:451 (1982). Electroporation of protoplasts and whole cells and tissues have also been described. Donn et al., In Abstracts of VIIth International Congress on Plant Cell and Tissue Culture IAPTC, A2-38, p53 (1990); D'Halluin et al., Plant Cell 4:1495-1505 (1992) and Spencer et al., Plant Mol. Biol. 24:51-61 (1994).

Following transformation of lettuce target tissues, expression of the above-described selectable marker genes allows for preferential selection of transformed cells, tissues and/or plants, using regeneration and selection methods now well known in the art.

The foregoing methods for transformation would typically be used for producing a transgenic cultivar. The transgenic cultivar could then be crossed, with another (non-transformed or transformed) cultivar, in order to produce a new transgenic cultivar. Alternatively, a genetic trait which has been engineered into a particular lettuce cultivar using the foregoing transformation techniques could be moved into another cultivar using traditional backcrossing techniques that are well known in the plant breeding arts. For example, a backcrossing approach could be used to move an engineered trait from a public, non-elite cultivar into an elite cultivar, or from a cultivar containing a foreign gene in its genome into a cultivar which does not contain that gene. As used herein, “crossing” can refer to a simple X by Y cross, or the process of backcrossing, depending on the context.

### Single or Multiple Gene Conversion

When the term lettuce plant is used in the context of the present invention, this also includes any single or multiple gene conversions of that plant. The terms single or multiple gene converted plant as used herein refers to those lettuce plants which are developed by a plant breeding technique called backcrossing wherein essentially all of the desired morphological and physiological characteristics of a cultivar are recovered in addition to the single or multiple gene(s) transferred into the cultivar via the backcrossing technique. Backcrossing methods can be used with the present invention to improve or introduce a characteristic into the cultivar. The term backcrossing as used herein refers to the repeated crossing of a hybrid progeny back to one of the parental lettuce plants, the recurrent parent, for that cultivar, i.e., backcrossing 1, 2, 3, 4, 5, 6, 7, 8, 9 or more times to the recurrent parent. The parental lettuce plant which contributes the gene for the desired characteristic is termed the nonrecurrent or donor parent. This terminology refers to the fact that the nonrecurrent parent is used one time in the backcross protocol and therefore does not recur. The parental lettuce plant to which the gene or genes from the nonrecurrent parent are transferred is known as the recurrent parent as it is used for several rounds in the backcrossing protocol (Fehr, W. R. et al. Principles of Cultivar Development—Theory and Technique pp. 261-286 (1987) and Pohelman and Sleper (1994)).

In a typical backcross protocol, the original cultivar of interest (recurrent parent) is crossed to a second cultivar (nonrecurrent parent) that carries the single or multiple gene(s) of interest to be transferred. The resulting progeny from this cross are then crossed again to the recurrent parent and the process is repeated until a lettuce plant is obtained wherein essentially all of the desired morphological and physiological characteristics of the recurrent parent are recovered in the converted plant, in addition to the single or multiple transferred gene(s) from the nonrecurrent parent as determined at the 5% significance level when grown in the same environmental conditions.

The selection of a suitable recurrent parent is an important step for a successful backcrossing procedure. The goal of a backcross protocol is to alter or substitute a single or multiple trait or characteristic in the original cultivar. To accomplish this, a single or multiple gene(s) of the recurrent cultivar is modified or substituted with the desired gene from the nonrecurrent parent, while retaining essentially all of the rest of the desired genetic, and therefore the desired physiological and morphological, constitution of the original cultivar. The choice of the particular nonrecurrent parent will depend on the purpose of the backcross; one of the major purposes is to add some commercially desirable, agronomically important trait to the plant. The exact backcrossing protocol will depend on the characteristic or trait being altered to determine an appropriate testing protocol. Although backcrossing methods are simplified when the characteristic being transferred is a dominant allele, a recessive allele may also be transferred. In this instance it may be necessary to introduce a test of the progeny to determine if the desired characteristic has been successfully transferred.

Many single or multiple gene traits have been identified that are not regularly selected for in the development of a new cultivar but that can be improved by backcrossing techniques. Single or multiple gene traits may or may not be transgenic, examples of these traits include but are not limited to, male sterility, waxy starch, herbicide resistance, resistance for bacterial, fungal, or viral disease, insect resistance, male fertility, enhanced nutritional quality, improved agronomic characteristics, industrial usage, yield stability and yield enhancement. These genes are generally inherited through the nucleus. Some known exceptions to this are the genes for male sterility, some of which are inherited cytoplasmically, but still act as single gene traits. Several of these single gene traits are described in U.S. Pat. Nos. 5,777,196; 5,948,957 and 5,969,212, the disclosures of which are specifically hereby incorporated by reference.

### Additional Methods for Genetic Engineering of Lettuce

In general, methods to transform, modify, edit, or alter plant endogenous genomic DNA include altering the plant native DNA sequence or a pre-existing transgenic sequence including regulatory elements, coding and non-coding sequences. These methods can be used, for example, to target nucleic acids to pre-engineered target recognition sequences in the genome. Such pre-engineered target sequences may be introduced by genome editing or modification. As an example, a genetically modified plant variety is generated using “custom” or engineered endonucleases such as meganucleases produced to modify plant genomes (see e.g., WO 2009/114321; Gao et al. (2010) Plant Journal 1:176-187). Another site-directed engineering method is through the use of zinc finger domain recognition coupled with the restriction properties of restriction enzyme. See e.g., Urnov, et al., (2010) Nat Rev Genet. 11(9):636-46; Shukla, et al., (2009) Nature 459 (7245):437-41. A transcription activator-like (TAL) effector-DNA modifying enzyme (TALE or TALEN) is also used to engineer changes in plant genome. See e.g., US20110145940, Cermak et al., (2011) Nucleic Acids Res. 39(12) and Boch et al., (2009), Science 326(5959): 1509-12. Site-specific modification of plant genomes can also be performed using the bacterial type II CRISPR (clustered regularly interspaced short palindromic repeats)/Cas (CRISPR-associated) system, as well as similar CRISPR related technologies. See e.g., Belhaj et al., (2013), Plant Methods 9: 39; The Cas9/guide RNA-based system allows targeted cleavage of genomic DNA guided by a customizable small noncoding RNA in plants (see e.g., WO 2015026883A1, incorporated herein by reference).

A genetic map can be generated that identifies the approximate chromosomal location of an integrated DNA molecule, for example via conventional restriction fragment length polymorphisms (RFLP), polymerase chain reaction (PCR) analysis, simple sequence repeats (SSR), and single nucleotide polymorphisms (SNP). For exemplary methodologies in this regard, see Glick and Thompson, Methods in Plant Molecular Biology and Biotechnology, pp. 269-284 (CRC Press, Boca Raton, 1993).

Wang et al. discuss “Large Scale Identification, Mapping and Genotyping of Single-Nucleotide Polymorphisms in the Human Genome”, Science (1998) 280:1077-1082, and similar capabilities are increasingly available for the radish genome. Map information concerning chromosomal location is useful for proprietary protection of a subject transgenic plant. If unauthorized propagation is undertaken and crosses made with other germplasm, the map of the integration region can be compared to similar maps for suspect plants to determine if the latter have a common parentage with the subject plant. Map comparisons could involve hybridizations, RFLP, PCR, SSR, sequencing or combinations thereof, all of which are conventional techniques. SNPs may also be used alone or in combination with other techniques.

### Tissue Culture

Further reproduction of lettuce plants which produce seeds having resistance to Fusarium wilt can occur by tissue culture and regeneration. Tissue culture of various tissues of lettuce and regeneration of plants therefrom is well known and widely published. For example, reference may be had to Komatsuda, T. et al., Crop Sci. 31:333-337 (1991); Stephens, P. A., et al., Theor. Appl. Genet. (1991) 82:633-635; Komatsuda, T. et al., Plant Cell, Tissue and Organ Culture, 28:103-113 (1992); Dhir, S. et al., Plant Cell Reports (1992) 11:285-289; Pandey, P. et al., Japan J. Breed. 42:1-5 (1992); and Shetty, K., et al., Plant Science 81:245-251 (1992); as well as U.S. Pat. No. 5,024,944 issued Jun. 18, 1991 to Collins et al., and U.S. Pat. No. 5,008,200 issued Apr. 16, 1991 to Ranch et al. Thus, another aspect of this invention is to provide cells which upon growth and differentiation produce lettuce plants having the physiological and morphological characteristics of lettuce plants which produce seeds having resistance to Fusarium wilt.

As used herein, the term “tissue culture” indicates a composition comprising isolated cells of the same or a different type or a collection of such cells organized into parts of a plant. Exemplary types of tissue cultures are protoplasts, calli, plant clumps, and plant cells that can generate tissue culture that are intact in plants or parts of plants, such as embryos, pollen, flowers, seeds, pods, leaves, stems, roots, root tips, anthers, and the like. Means for preparing and maintaining plant tissue culture are well known in the art. By way of example, a tissue culture comprising organs has been used to produce regenerated plants. U.S. Pat. Nos. 5,959,185; 5,973,234 and 5,977,445 describe certain techniques, the disclosures of which are incorporated herein by reference.

As used herein, the term “plant” includes plant cells, plant protoplasts, plant cells of tissue culture from which lettuce plants can be regenerated, plant calli, plant clumps, and plant cells that are intact in plants or parts of plants, such as pollen, flowers, embryos, ovules, seeds, pods, leaves, stems, pistils, anthers and the like.

Duncan, et al., Planta 165:322-332 (1985) reflects that 97% of the plants cultured that produced callus were capable of plant regeneration. Subsequent experiments with both cultivars and hybrids produced 91% regenerable callus that produced plants. In a further study in 1988, Songstad, et al., Plant Cell Reports 7:262-265 (1988), reports several media additions that enhance regenerability of callus of two cultivars. Other published reports also indicated that “non-traditional” tissues are capable of producing somatic embryogenesis and plant regeneration. K. P. Rao et al., Maize Genetics Cooperation Newsletter, 60:64-65 (1986), refers to somatic embryogenesis from glume callus cultures and B. V. Conger, et al., Plant Cell Reports, 6:345-347 (1987) indicates somatic embryogenesis from the tissue cultures of corn leaf segments. Thus, it is clear from the literature that the state of the art is such that these methods of obtaining plants are routinely used and have a very high rate of success.

### Additional Breeding Methods

There are numerous steps in the development of any novel, desirable plant germplasm. Plant breeding begins with the analysis and definition of problems and weaknesses of the current germplasm, the establishment of program goals, and the definition of specific breeding objectives. The next step is selection of germplasm that possess the traits to meet the program goals. The goal is to combine in a single variety an improved combination of desirable traits from the parental germplasm. These important traits may include higher seed yield, resistance to diseases and insects, better stems and roots, resistance to low temperatures, resistance to herbicides, and better agronomic characteristics on grain quality.

Choice of breeding or selection methods depends on the mode of plant reproduction, the heritability of the trait(s) being improved, and the type of cultivar used commercially (e.g., F1 hybrid cultivar, pureline cultivar, etc.). For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection, or a combination of these methods.

The complexity of inheritance influences choice of the breeding method. Backcross breeding is used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Each breeding program should include a periodic, objective evaluation of the efficiency of the breeding procedure. Evaluation criteria vary depending on the goal and objectives but should include gain from selection per year based on comparisons to an appropriate standard, overall value of the advanced breeding lines, and number of successful cultivars produced per unit of input (e.g., per year, per dollar expended, etc.).

Promising advanced breeding lines are thoroughly tested and compared to appropriate standards in environments representative of the commercial target area(s) for three or more years. The best lines are candidates for new commercial cultivars; those still deficient in a few traits may be used as parents to produce new populations for further selection.

These processes, which lead to the final step of marketing and distribution, usually take from 8 to 12 years from the time the first cross is made and may rely on the development of improved breeding lines as precursors. Therefore, development of new cultivars is a time-consuming process that requires precise forward planning, efficient use of resources, and a minimum of changes in direction.

A most difficult task is the identification of individuals that are genetically superior, because for most traits the true genotypic value is masked by other confounding plant traits or environmental factors. One method of identifying a superior plant is to observe its performance relative to other experimental plants and to a widely grown standard cultivar. If a single observation is inconclusive, replicated observations provide a better estimate of its genetic worth.

The goal of lettuce plant breeding is to develop new, unique and superior lettuce cultivars and hybrids. The breeder initially selects and crosses two or more parental lines, followed by self-pollination and selection, producing many new genetic combinations. The breeder can theoretically generate billions of different genetic combinations via crossing, selfing and mutations. The breeder has no direct control at the cellular level. Therefore, two breeders will never develop the same line, or even very similar lines, having the same lettuce traits.

Each year, the plant breeder selects the germplasm to advance to the next generation. This germplasm is grown under unique and different geographical, climatic and soil conditions, and further selections are then made, during and at the end of the growing season. The cultivars which are developed are unpredictable. This unpredictability is because the breeder's selection occurs in unique environments, with no control at the DNA level (using conventional breeding procedures), and with millions of different possible genetic combinations being generated. A breeder of ordinary skill in the art cannot predict the final resulting lines he develops, except possibly in a very gross and general fashion. The same breeder cannot produce the same cultivar twice by using the exact same original parents and the same selection techniques. This unpredictability results in the expenditure of large amounts of research monies to develop superior new lettuce cultivars.

The development of new lettuce cultivars requires the development and selection of lettuce varieties, the crossing of these varieties and selection of superior hybrid crosses. The hybrid seed is produced by manual crosses between selected male-fertile parents or by using male sterility systems. These hybrids are selected for certain single gene traits such as semi-dwarf plant type, pubescence, awns, and apiculus color which indicate that the seed is truly a hybrid. Additional data on parental lines, as well as the phenotype of the hybrid, influence the breeder's decision whether to continue with the specific hybrid cross.

Pedigree breeding and recurrent selection breeding methods are used to develop cultivars from breeding populations. Breeding programs combine desirable traits from two or more cultivars or various broad-based sources into breeding pools from which cultivars are developed by selfing and selection of desired phenotypes. The new cultivars are evaluated to determine which have commercial potential.

Pedigree breeding is used commonly for the improvement of self-pollinating crops. Two parents which possess favorable, complementary traits are crossed to produce an F₁. An F₂ population is produced by selfing one or several F₁'s. Selection of the best individuals may begin in the F₂ population; then, beginning in the F₃, the best individuals in the best families are selected. Replicated testing of families can begin in the F₄ generation to improve the effectiveness of selection for traits with low heritability. At an advanced stage of inbreeding (i.e., F₆ and F₇), the best lines or mixtures of phenotypically similar lines are tested for potential release as new cultivars.

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Backcross breeding has been used to transfer genes for a simply inherited, highly heritable trait into a desirable homozygous cultivar or inbred line which is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

In a multiple-seed procedure, lettuce breeders commonly harvest one or more seeds from each plant in a population and thresh them together to form a bulk. Part of the bulk is used to plant the next generation, and part is put in reserve. The procedure has been referred to as modified single-seed descent or the pod-bulk technique.

The multiple-seed procedure has been used to save labor at harvest. It is considerably faster to thresh panicles with a machine than to remove one seed from each by hand for the single-seed procedure. The multiple-seed procedure also makes it possible to plant the same number of seeds of a population for each generation of inbreeding. Enough seeds are harvested to make up for those plants that did not germinate or produce seed.

Mutation breeding is another method of introducing new traits into lettuce lines. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), alkylating agents (such as sulfur mustards, nitrogen mustards, epoxides, ethyleneamines, sulfates, sulfonates, sulfones, or lactones), azide, hydroxylamine, nitrous acid or acridines. Once a desired phenotype is observed the genetic mutation responsible for that trait may then be incorporated into existing germplasm by traditional breeding techniques. The use of mutagenic agents to create genetic diversity in lettuces as well as useful lettuce mutants is known in the art. Details of mutation breeding can be found in Principles of Cultivar Development by Fehr, Macmillan Publishing Company, 1993.

The production of double haploids can also be used for the development of homozygous varieties in a breeding program. Double haploids are produced by doubling a set of chromosomes from a heterozygous plant to produce a completely homozygous individual. For example, see Wan et al., Theor. Appl. Genet., 77:889-892, 1989.

Descriptions of other breeding methods that are commonly used for different traits and crops can be found in several reference books (e.g., Principles of Plant Breeding John Wiley and Son, pp. 115-161, 1960; Allard, 1960; Simmonds, 1979; Sneep et al., 1979; Fehr, 1987; Carrots and Related Vegetable Umbelliferae, Rubatzky, V. E., et al., 1999).

### Genetic Analysis

In addition to phenotypic observations, the genotype of a plant can also be examined. There are many laboratory-based techniques available for the analysis, comparison and characterization of plant genotype; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length polymorphisms (AFLPs), Simple Sequence Repeats (SSRs—which are also referred to as Microsatellites), and Single Nucleotide Polymorphisms (SNPs).

Isozyme Electrophoresis and RFLPs have been widely used to determine genetic composition. Shoemaker and Olsen (Molecular Linkage Map of Soybean (Glycine max), pp. 6.131-6.138 in S. J. O'Brien (ed.) Genetic Maps: Locus Maps of Complex Genomes, Cold Spring Harbor Laboratory Press, Cold Spring Harbor, NY (1993)) developed a molecular genetic linkage map that consisted of 25 linkage groups with about 365 RFLP, 11 RAPD, three classical markers, and four isozyme loci. See also, Shoemaker, R. C., RFLP Map of Soybean, pp. 299-309, in Phillips, R. L. and Vasil, I. K. (eds.), DNA-Based Markers in Plants, Kluwer Academic Press, Dordrecht, the Netherlands (1994).

The invention further provides a method of determining the genotype of a lettuce plant having resistance to Fusarium wilt which produces seeds having resistance to Fusarium wilt, or a first-generation progeny thereof, which may comprise obtaining a sample of nucleic acids from said plant and detecting in said nucleic acids a plurality of polymorphisms. This method may additionally comprise the step of storing the results of detecting the plurality of polymorphisms on a computer readable medium. The plurality of polymorphisms is indicative of and/or give rise to the expression of the morphological and physiological characteristics of a lettuce plant having resistance to Fusarium wilt, which produces seeds having resistance to Fusarium wilt, such seeds producing plants having resistance to Fusarium wilt.

With any of the genotyping techniques mentioned herein, polymorphisms may be detected when the genotype and/or sequence of the plant of interest is compared to the genotype and/or sequence of one or more reference plants. The polymorphism revealed by these techniques may be used to establish links between genotype and phenotype. The polymorphisms may thus be used to predict or identify certain phenotypic characteristics, individuals, or even species. The polymorphisms are generally called markers. It is common practice for the skilled artisan to apply molecular DNA techniques for generating polymorphisms and creating markers. The polymorphisms of this invention may be provided in a variety of mediums to facilitate use, e.g. a database or computer readable medium, which may also contain descriptive annotations in a form that allows a skilled artisan to examine or query the polymorphisms and obtain useful information.

SSR technology is currently the most efficient and practical marker technology; more marker loci can be routinely used and more alleles per marker locus can be found using SSRs in comparison to RFLPs. For example, Diwan and Cregan described a highly polymorphic microsatellite locus in soybean with as many as 26 alleles. (Diwan, N. and Cregan, P. B., Theor. Appl. Genet. 95:22-225, 1997.) Single Nucleotide Polymorphisms (SNPs) may also be used to identify the unique genetic composition of the invention and progeny varieties retaining that unique genetic composition. Various molecular marker techniques may be used in combination to enhance overall resolution.

Molecular markers, which include markers identified through the use of techniques such as Isozyme Electrophoresis, RFLPs, RAPDs, AP-PCR, DAF, SCARs, AFLPs, SSRs, and SNPs, may be used in plant breeding. One use of molecular markers is Quantitative Trait Loci (QTL) mapping. QTL mapping is the use of markers which are known to be closely linked to alleles that have measurable effects on a quantitative trait. Selection in the breeding process is based upon the accumulation of markers linked to the positive effecting alleles and/or the elimination of the markers linked to the negative effecting alleles from the plant's genome.

Molecular markers can also be used during the breeding process for the selection of qualitative traits. For example, markers closely linked to alleles or markers containing sequences within the actual alleles of interest can be used to select plants that contain the alleles of interest during a backcrossing breeding program. The markers can also be used to select toward the genome of the recurrent parent and against the markers of the donor parent. This procedure attempts to minimize the amount of genome from the donor parent that remains in the selected plants. It can also be used to reduce the number of crosses back to the recurrent parent needed in a backcrossing program. The use of molecular markers in the selection process is often called genetic marker enhanced selection or marker-assisted selection. Flanking markers that are tightly linked to target genes can be used for selection and are sometimes more efficient than direct selection for the target genes. Use of flanking markers on either side of the locus of interest during marker assisted selection increases the probability that the desired gene is selected. Molecular markers may also be used to identify and exclude certain sources of germplasm as parental varieties or ancestors of a plant by providing a means of tracking genetic profiles through crosses.

Particular markers used for these purposes are not limited to the set of markers disclosed herein but may include any type of marker and marker profile which provides a means of distinguishing varieties. In addition to being used for identification of lettuce plants having resistance to Fusarium wilt, which produce seeds having resistance to Fusarium wilt, a hybrid produced through the use of lettuce plants having resistance to Fusarium wilt, and the identification or verification of pedigree for progeny plants produced through the use of lettuce plants which produce seeds having resistance to Fusarium wilt, which in turn produce lettuce plants having resistance to Fusarium wilt, a genetic marker profile is also useful in developing a locus conversion of lettuce plants which produce seeds having resistance to Fusarium wilt.

Means of performing genetic marker profiles using SNP and SSR polymorphisms are well known in the art. SNPs are genetic markers based on a polymorphism in a single nucleotide. A marker system based on SNPs can be highly informative in linkage analysis relative to other marker systems in that multiple alleles may be present.

Proper testing should detect any major faults and establish the level of superiority or improvement over current cultivars. In addition to showing superior performance, there must be a demand for a new cultivar that is compatible with industry standards or which creates a new market. The introduction of a new cultivar will incur additional costs to the seed producer, the grower, processor and consumer; for special advertising and marketing, altered seed and commercial production practices, and new product utilization. The testing preceding release of a new cultivar should take into consideration research and development costs as well as technical superiority of the final cultivar. For seed-propagated cultivars, it must be feasible to produce seed easily and economically.

Lettuce varieties which produce seeds having resistance to Fusarium wilt of the present invention can also be used for transformation where exogenous genes are introduced and expressed by the variety. Genetic variants created either through traditional breeding methods using a line which produces seeds having resistance to Fusarium wilt or through transformation of a lettuce line having resistance to Fusarium wilt which produces seeds having resistance to Fusarium wilt, which in turn produce lettuce plants having resistance to Fusarium wilt by any of a number of protocols known to those of skill in the art are intended to be within the scope of this invention.

The following describes breeding methods that may be used with a lettuce plant having resistance to Fusarium wilt, which produces seeds having resistance to Fusarium wilt, which in turn produces lettuce plants having resistance to Fusarium wilt in the development of further lettuce plants. One such embodiment is a method for developing a progeny lettuce plant in a lettuce plant breeding program comprising: obtaining a lettuce plant, or a part thereof, which produces seeds having resistance to Fusarium wilt, utilizing said plant or plant part as a source of breeding material and selecting a progeny plant which produces seeds with molecular markers in common with lettuce plants which produce seeds having resistance to Fusarium wilt. Breeding steps that may be used in the lettuce plant breeding program include pedigree breeding, back crossing, mutation breeding, and recurrent selection. In conjunction with these steps, techniques such as RFLP-enhanced selection, genetic marker enhanced selection (for example SSR markers) and the making of double haploids may be utilized. Double haploids are produced by the doubling of a set of chromosomes (1 N) from a heterozygous plant to produce a completely homozygous individual. For example, see Wan et al., “Efficient Production of Doubled Haploid Plants Through Colchicine Treatment of Anther-Derived Maize Callus”, Theoretical and Applied Genetics, 77:889-892, 1989 and U.S. Pat. No. 7,135,615.

One of ordinary skills in the art of plant breeding would be to know how to evaluate the traits of two plant varieties to determine if there is no significant difference between the two traits expressed by those varieties. For example, see Fehr and Walt, Principles of Cultivar Development, p 261-286 (1987). Thus, the invention includes lettuce plants which produces seeds having resistance to Fusarium wilt, progeny lettuce plants so that said progeny lettuce plants are not significantly different for said traits than a lettuce plant which produces seeds having resistance to Fusarium wilt, which in turn produce lettuce plants having resistance to Fusarium wilt. Using techniques described herein, molecular markers may be used to identify said progeny plant as a plant which produces seeds having resistance to Fusarium wilt. Mean trait values may be used to determine whether trait differences are significant, and preferably the traits are measured on plants grown under the same environmental conditions. Once such a variety is developed its value is substantial since it is important to advance the germplasm base as a whole in order to maintain or improve traits such as yield, disease resistance, pest resistance, and plant performance in extreme environmental conditions.

Progeny of lettuce plants which produce seeds having resistance to Fusarium wilt may also be characterized through their filial relationship with lettuce plants having resistance to Fusarium wilt which produce seeds having resistance to Fusarium wilt as for example, being within a certain number of breeding crosses of lettuce plants which produce seeds having resistance to Fusarium wilt. A breeding cross is a cross made to introduce new genetics into the progeny, and is distinguished from a cross, such as a self or a sib cross, made to select among existing genetic alleles. The lower the number of breeding crosses in the pedigree, the closer the relationship between lettuce having resistance to Fusarium wilt plants which produce seeds having resistance to Fusarium wilt and its progeny. For example, progeny produced by the methods described herein may be within 1, 2, 3, 4 or 5 or more breeding crosses of lettuce plants having resistance to Fusarium wilt which produce seeds having resistance to Fusarium wilt, which in turn produces lettuce plants having resistance to Fusarium wilt.

### Deposit Information

Deposits of the Southwest Genetics, LLC lettuce lines designated ADRIAN, MICKEY, BALBOA, and PAULIE, having resistance to Fusarium wilt, disclosed above and recited in the claims, have been made with the American Type Culture Collection (ATCC), 10801 University Boulevard, Manassas, VA 20110, under the Budapest Treaty. The date of the deposits was Jul. 21, 2023. Deposits of 25 packets of 25 seeds in each packet for each of the designated lettuce lines was taken from the same deposit maintained by Southwest Genetics, LLC since prior to the filing date of this application. All restrictions will be irrevocably removed upon granting of a patent, and the deposits are intended to meet all requirements of 37 C.F.R. §§ 1.801-1.809. The ATCC accession numbers are PTA-127613, PTA-127616, PTA-127614, and PTA-127615, respectively. Access to the deposits will be available during the pendency of this application to persons determined by the Commissioner of Patents and Trademarks to be entitled thereto under 37 C.F.R. § 1.14 and 35 U.S.C. § 122. Upon allowance of any claims in this application, all restrictions on the availability to the public of the lettuce lines will be irrevocably removed. The deposits will be maintained in the depository for a period of 30 years, or 5 years after the last request, or the effective life of the patent, whichever is longer, and will be replaced, if necessary, during this period.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions, and sub-combinations as are within their true spirit and scope.

What is claimed is:

1. A lettuce seed having a resistance to Fusarium wilt.

2. A seed of claim 1, wherein a representative sample of seed having resistance to Fusarium wilt was deposited under ATCC Accession No. PTA-127613, PTA-127616, PTA-127614, and PTA-127615.

3. A lettuce plant having resistance to Fusarium wilt produced by growing the seed of claim 2, or a plant part thereof.

4. A tissue culture produced from protoplasts or cells from the plant part of the lettuce plant of claim 3, wherein said plant part is selected from the group consisting of leaf, pollen, ovule, embryo, cotyledon, hypocotyl, meristematic cell, root, root tip, pistil, anther, flower, seed, pedicel, stem, and petiole.

5. A lettuce plant having resistance to Fusarium wilt regenerated from the tissue culture of claim 4.

6. A method for producing a lettuce seed, said method comprising crossing two lettuce plants and harvesting the resultant lettuce seed, wherein at least one lettuce plant is the lettuce plant of claim 3.

7. A lettuce seed produced by the method of claim 6.

8. A lettuce plant produced by growing said seed of claim 7, or a plant part thereof.

9. The method of claim 6, wherein at least one of said lettuce plants is transgenic.

10. A method of producing an herbicide resistant lettuce plant, wherein said method comprises introducing a gene conferring herbicide resistance into the lettuce plant of claim 3.

11. An herbicide resistant lettuce plant produced by the method of claim 10.

12. A method of producing a pest or insect resistant lettuce plant, wherein said method comprises introducing a gene conferring pest or insect resistance into the lettuce plant of claim 3.

13. A pest or insect resistant lettuce plant produced by the method of claim 12.

14. A method of producing a disease resistant lettuce plant, wherein said method comprises introducing a gene which confers disease resistance into the lettuce plant of claim 3.

15. A disease resistant lettuce plant produced by the method of claim 14.

16. A method of producing a lettuce plant with a desired trait, wherein the method comprises introducing a gene mutation via chemical mutagenesis into the lettuce plant of claim 3.

17. A lettuce plant produced by the method of claim 16.

18. A method of introducing a desired trait into a lettuce plant of claim 3, wherein the method comprises:
a. crossing a lettuce plant of claim 3 with a plant of another lettuce cultivar that comprises a desired trait, wherein the desired trait is selected from the group consisting of low herbicide resistance, insect resistance, modified shape, or color, and resistance to bacterial disease, fungal disease or viral disease;
b. selecting one or more progeny plants that have the desired trait to produce selected progeny plants;
c. backcrossing the selected progeny plants with either parent cultivar to produce backcross progeny plants;
d. selecting for backcross progeny plants that have the desired trait and resistance to Fusarium wilt; and
e. repeating steps (c) and (d) two or more times in succession to produce selected third or higher backcross progeny plants that comprise the desired trait and produce seeds having resistance to Fusarium wilt.

19. A lettuce plant produced by the method of claim 18, wherein the lettuce plant has the desired trait and resistance to Fusarium wilt.

20. A method of producing a genetically modified lettuce plant, wherein the method comprises mutation, transformation, gene conversion, genome editing, RNA interference or gene silencing of the plant of claim 3.

21. A genetically modified lettuce plant produced by the method of claim 20, wherein the plant comprises the genetic modification and produces seed having resistance to Fusarium wilt.
